(19) United States
(12) Patent Application Publication
(10) Pub. No.: US 2026/0060730 A1
(43) Pub. Date: Mar. 5, 2026

(54) ORTHOPEDIC FIXATION ASSEMBLY WITH HELICOID PIEZOELECTRIC INTERFACE

(71) Applicants: Ziev Moses, Needham, MA (US); Kevin Chappuis, Saugus, MA (US); Luke Diehl, Medford, MA (US); Kevin T. Foley, Germantown, TN (US)

(72) Inventors: Ziev Moses, Needham, MA (US); Kevin Chappuis, Saugus, MA (US); Luke Diehl, Medford, MA (US); Kevin T. Foley, Germantown, TN (US)

(21) Appl. No.: 19/379,210

(22) Filed: Nov. 4, 2025

Related U.S. Application Data
(63) Continuation-in-part of application No. 18/900,021, filed on Sep. 27, 2024.
Application No. 18/900,021 is a continuation-in-part of application No. 18/308,406, filed on Apr. 27, 2023.
Application No. 18/900,021 is a continuation-in-part of application No. 18/308,455, filed on Apr. 27, 2023.
(63) Continuation-in-part of application No. 18/308,406, filed on Apr. 27, 2023.
(63) Continuation-in-part of application No. 18/308,455, filed on Apr. 27, 2023.
(60) Provisional application No. 63/335,343, filed on Apr. 27, 2022.
(60) Provisional application No. 63/335,343, filed on Apr. 27, 2022.
(60) Provisional application No. 63/396,019, filed on Aug. 8, 2022.
(60) Provisional application No. 63/723,713, filed on Nov. 22, 2024.

(51) Int. Cl. A61B 17/86; A61B 17/00

(52) U.S. Cl. CPC A61B 17/863; A61B 17/8605; A61B 17/864; A61B 17/8685; A61B 17/8695; A61B 2017/00526; A61B 2017/00831; A61B 2017/8655

(57) ABSTRACT

An orthopedic fixation assembly and a kit for forming same. The orthopedic fixation assembly comprises: an elongated body comprising a first lateral component having a first inner surface, and a second lateral component having a second inner surface, the second inner surface facing and conformal with the first inner surface and defining an interface therewith, the interface defining a helicoid shape about a line extending through the elongated body; and a piezoelectric material at the interface. The kit to make an orthopedic fixation assembly comprises a first lateral component having a first inner surface defining a first helicoid shape; a second lateral component having a second inner surface defining a second helicoid shape, wherein the first lateral component and the second lateral component are configured to be assembled together to form an elongated body, wherein, in the elongated body, the second inner surface faces the first inner surface and the first helicoid shape and the second helicoid shape define an interface therebetween, the interface defining an interfacial helicoid shape about a line extending through the elongated body; and a piezoelectric material configured to be placed in the elongated body at the interface.

### CROSS-REFERENCE TO RELATED APPLICATIONS

This application claims priority to U.S. Provisional Patent Ser. No. 63/723,713 entitled “PIEZOELECTRIC SACROILIAC JOIN FUSION IMPLANT ASSEMBLY AND KIT FOR MAKING SAME” filed on Nov. 22, 2024. This application further is a continuation-in-part of U.S. Utility patent application Ser. No. 18/900,021 entitled “PIEZOELECTRIC SPINAL IMPLANT ASSEMBLY HAVING ENDPLATES WITH NON-PLANAR SURFACES” filed on Sep. 27, 2024, which is a continuation-in-part of U.S. Utility patent application Ser. No. 18/308,406 entitled “PIEZOELECTRIC SPINAL IMPLANT AND METHODS OF MAKING AND USING SAME” filed on Apr. 27, 2023, which claims priority to U.S. Provisional Patent Application Ser. No. 63/335,343 entitled “PIEZOELECTRIC SPINAL IMPLANT AND METHODS OF MAKING AND USING SAME,” and is further a continuation-in-part of U.S. Utility patent application Ser. No. 18/308,455 entitled “PIEZOELECTRIC ORTHOPEDIC IMPLANT AND METHODOLOGY” filed on Apr. 27, 2023, which claims priority to two U.S. Provisional Patent Applications, namely, 63/335,343 entitled “PIEZOELECTRIC SPINAL IMPLANT AND METHODS OF MAKING AND USING SAME” filed on Apr. 27, 2022, and U.S. Provisional Patent Application Ser. No. 63/396,019 entitled “PIEZOELECTRIC ORTHOPEDIC IMPLANT AND METHODOLOGY,” and filed on Aug. 8, 2022. This application is further a continuation-in-part of U.S. Utility patent application Ser. No. 18/308,406 entitled “PIEZOELECTRIC SPINAL IMPLANT AND METHODS OF MAKING AND USING SAME” filed on Apr. 27, 2023, which claims priority to U.S. Provisional Patent Application Ser. No. 63/335,343 entitled “PIEZOELECTRIC SPINAL IMPLANT AND METHODS OF MAKING AND USING SAME” filed on Apr. 27, 2022. This application is further a continuation-in-part of U.S. Utility patent application Ser. No. 18/308,455 entitled “PIEZOELECTRIC ORTHOPEDIC IMPLANT AND METHODOLOGY” filed on Apr. 27, 2023, which claims priority to two U.S. Provisional Patent Applications, namely, 63/335,343 entitled “PIEZOELECTRIC SPINAL IMPLANT AND METHODS OF MAKING AND USING SAME” filed on Apr. 27, 2022, and U.S. Provisional Patent Application Ser. No. 63/396,019 entitled “PIEZOELECTRIC ORTHOPEDIC IMPLANT AND METHODOLOGY,” and filed on Aug. 8, 2022. The disclosures of the prior applications are incorporated herein by reference in their entireties.

### FIELD

The present application relates to implants orthopedic fixation devices.

### BACKGROUND

Orthopedic fixation devices are commonly used to stabilize bone segments in the treatment of fractures, fusions, and reconstructive procedures. These devices, which include screws, plates, rods, nails, pins, and prosthetic stems, are designed to maintain alignment and provide mechanical support during healing. In many applications, fixation devices are assembled from multiple components that must remain securely joined under physiological loads. However, fixation devices can be subject to forces that may cause components to loosen, separate, or migrate from their intended position. For example, screws or nails may back out, modular components may disengage, or assembled parts may experience relative movement, particularly under repetitive loading or in anatomically challenging locations. Such events can compromise the stability of the fixation construct and may necessitate additional intervention. Accordingly, there is a need for orthopedic fixation devices with improved resistance to loosening, migration, and component separation.

### BRIEF DESCRIPTION OF THE FIGURES

Further embodiments of the present application can be understood with reference to the appended figures.

FIG. 1A illustrates an implant environment including sacroiliac (SI) joint screws anchored into hip bones.

FIG. 1B shows an implant environment with fractured humerus bone segments repaired using lag screws and trauma screws.

FIG. 1C depicts an implant environment with a fixation plate bridging a tibia-fibula fracture, affixed by multiple screws.

FIG. 1D presents an environment with a pedicle screw assembly stabilizing adjacent vertebrae.

FIG. 1E illustrates an implant environment with an intramedullary nail extending through the length of a tibia or femur.

FIG. 1F shows an implant environment with multiple bone pins spanning bones in the foot.

FIGS. 2A, 2B, and 2C provide side elevational views of arthroplasty assemblies, including femoral and tibial components and prosthetic stems.

FIGS. 3A and 3B are x-ray images of a total hip prosthesis assembly including an articular head and prosthetic stem.

FIGS. 4A and 4B show perspective views of an orthopedic fixation assembly comprising first and second lateral components.

FIGS. 4C and 4D are exploded views of a total prosthesis assembly including an orthopedic fixation assembly and an articulated head.

FIGS. 5A and 5B illustrate an orthopedic fixation assembly with threaded holes and a screw for securing lateral components.

FIG. 5C shows a kit for assembling the orthopedic fixation assembly of FIGS. 5A and 5B.

FIG. 6A depicts a rod-shaped orthopedic fixation assembly with a smooth outer surface and a fastener cap.

FIG. 6B shows a kit for assembling the orthopedic fixation assembly of FIG. 6A.

FIGS. 7A and 7B illustrate a rod-shaped orthopedic fixation assembly with a cannulated lumen for guided implantation.

FIG. 7C shows a kit for assembling the orthopedic fixation assembly of FIGS. 7A and 7B, including piezoelectric material and grooves for a guidewire lumen.

FIG. 8 presents a screw-shaped orthopedic fixation assembly with a threaded outer surface and a splined fastener cap.

FIGS. 9A and 9B show a screw-shaped orthopedic fixation assembly with fastener caps at both proximal and distal ends.

FIGS. 10A and 10B provide perspective views of a screw-shaped orthopedic fixation assembly with a washer portion.

FIG. 10C shows a kit for assembling the orthopedic fixation assembly of FIGS. 10A and 10B.

FIG. 10D is an exploded view of the orthopedic fixation assembly of FIGS. 10A-10C, showing lateral components, piezoelectric material, and grooves for a guidewire lumen.

FIGS. 11A, 11B, and 11C illustrate screw-shaped orthopedic fixation assemblies with interfaces of varying numbers of revolutions.

FIG. 12 provides cross-sectional views of an orthopedic fixation assembly at different locations, showing the orientation of lateral components and piezoelectric material.

FIGS. 13A and 13B illustrate the behavior of an orthopedic fixation assembly under loaded and unloaded conditions, showing charge distribution and electron flow between lateral components.

### DETAILED DESCRIPTION

Embodiments are directed to orthopedic implants that utilize an assembly of two or more electrically isolated lateral components to sandwich a piezoelectric component, creating a dynamically actuated piezoelectric implantable device. Orthopedic fixation assemblies and kits for forming same are provided. The orthopedic fixation assembly comprises: an elongated body comprising a first lateral component having a first inner surface, and a second lateral component having a second inner surface, the second inner surface facing and conformal with the first inner surface and defining an interface therewith, the interface defining a helicoid shape about a line extending through the elongated body; and a piezoelectric material at the interface.

Embodiments may incorporate a variety of helicoid shapes ranging from slightly less than a full revolution to multiple revolutions, including fractions of revolutions such as 0.8 revolution, 1.5 revolutions, 2 revolutions, 4 revolutions, applicable for screws, stems, nails, dowels/rods or other implant forms.

According to some embodiments, the first lateral component and the second lateral component both include an electrically conductive material. In another aspect, the orthopedic fixation assembly further includes a fastener to secure the first lateral component and the second lateral component to one another. In some embodiments, the first lateral component and the second lateral component are configured for self-interlocking, for example, using a snap fit or a press fit. The fastener may include an electrically insulating component or may itself be the electrically insulating component. In other cases, the fastener is threaded or arranged for press fit fastening and may additionally be a pin. Some embodiments include a fastener formed from both a metal component and an electrically insulating component, where the insulating component can be PEEK. Other configurations provide an electrically conductive threaded component combined with an electrically isolating washer, so that the washer electrically insulates the threaded component from the first and second lateral components.

According to some embodiments, the piezoelectric material comprises one or more piezoelectric elements, and in certain forms, the piezoelectric material corresponds to one or more sheets located between the first inner surface and the second inner surface. In some instances, the piezoelectric sheet spans the entirety of the interface between the first inner surface and the second inner surface. In some instances, the piezoelectric sheet spans one or more parts but not the entirety of the interface between the first inner surface and the second inner surface. Some embodiments feature an elongated body that is substantially cylindrical, although an elongated body according to embodiments may have any shape. In some cases, the elongated body serves as a prosthetic stem of a larger orthopedic fixation assembly, which further includes an articulated head such as a femoral head or a humeral head.

In some embodiments, the piezoelectric material may correspond to a material exhibiting strong piezoelectric properties, such as polyvinylidene fluoride (PVDF), polyvinylidene fluoride-trifluoroethylene (PVDF-TrFE), or other suitable piezoelectric polymers. Alternatively, the piezoelectric layer may comprise a composite or matrix material that includes piezoelectric fillers, such as piezoelectric ceramic fibers, rods, or particles dispersed within a polymeric or elastomeric matrix. Suitable piezoelectric fillers may include lead zirconate titanate (PZT), barium titanate, or other piezoelectric ceramics, as well as piezoelectric single-crystal fibers or whiskers. The use of a matrix with embedded piezoelectric fibers or particles allows for tailored mechanical and electrical properties, improved conformability to the interface between lateral components, and enhanced durability under cyclic loading conditions.

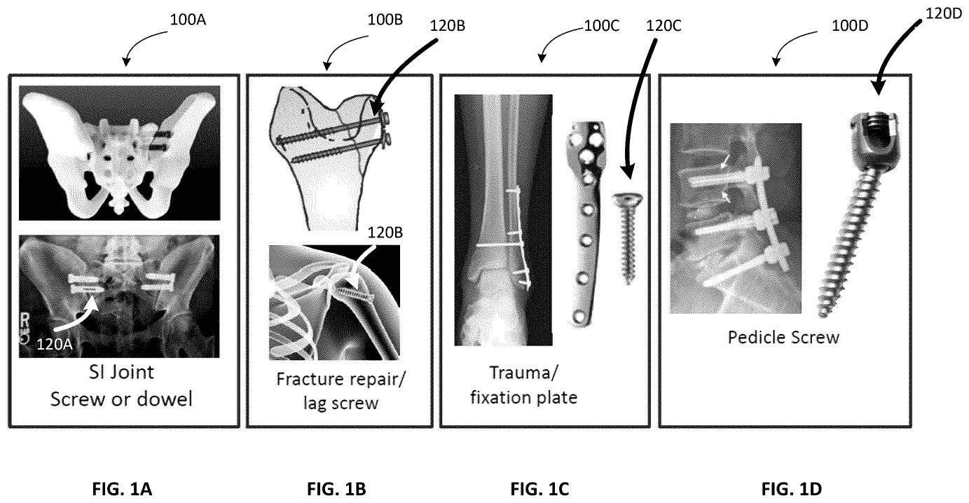

The assembly may be implemented as an orthopedic screw, nail, or arthroplasty stem, with all configurations utilizing a piezoelectric mechanism to stimulate bone growth. Example applications for these embodiments are provided in FIGS. 1A-IF, which detail clinical environments where the orthopedic fixation assembly may be implemented.

Some embodiments include providing an orthopedic fixation assembly such as screws, pins, dowels, or rods, or any orthopedic implant assemblies designed to traverse bone and/or bone marrow after implantation and containing a piezoelectric material to encourage bone formation at the site. Orthopedic fixation assemblies according to embodiments may have inner surfaces that twist about the longitudinal axis as the assembly extends, with the angle between facing surfaces rotating between various longitudinal positions.

Certain aspects provide continuous inner surfaces forming a three-dimensional spiral along the axis; other aspects provide discontinuous inner surfaces, with discrete facing regions and corresponding rotations of the normal vectors. As used here, an orthopedic fixation assembly described as a “nail,” “pin,” or “dowel” is to have a non-threaded outer surface, while a “screw” embodiment is to have a threaded outer surface. orthopedic fixation assemblies according to these embodiments may additionally feature either smooth or porous outer surfaces, with porous surfaces used to promote further osteogenesis on the implant's external surface.

By way of example, the lateral components that mate or conform with each other may be manufactured from metal-such as titanium-which may be either smooth or porous, and configured for optimal osteointegration, electrical performance, mechanical fixation, and modularity, as detailed throughout the figures below and clinical applications described herein.

Any of the implantation environments depicted in FIGS. 1A-ID may incorporate orthopedic fixation assemblies according to some embodiments, each of which is configured with piezoelectric properties adapted to stimulate osteogenesis at the site of implantation. The figures themselves are intended to illustrate the breadth of possible clinical scenarios for such orthopedic fixation assemblies, even though specific structural details of the orthopedic fixation assemblies themselves are not always shown within these figure sets; use cases are not restricted only to these examples.

Let us now refer to FIGS. 1A-1D.

FIG. 1A presents implant environments 100A, where sacroiliac (SI) joint screws 120A are anchored into the hip bones, thereby providing stabilization across the sacroiliac joint.

FIG. 1B illustrates implant environments 100B, which feature fractured humerus bone segments repaired with screws 120B. These include both lag screws, shown in the upper portion of FIG. 1B, that compress fracture fragments together, as well as regular trauma screws, shown at the bottom of FIG. 1B, that secure bone fragments in alignment.

FIG. 1C depicts implant environment 100C along with an exploded view of a fixation plate 120C, which is utilized to bridge and stabilize a tibia-fibula fracture, with the plate 120C affixed via multiple screws across the fracture interface.

FIG. 1D highlights environment 100D and provides a view of a pedicle screw assembly 120D, wherein a set of pedicle screws are inserted to stabilize adjacent vertebrae.

In collectively viewing FIGS. 1A-1D, it is evident that SI joint fixation routinely employs screws, while additional options include smooth dowel-type implants, which may be designed with faceted cross-sections, such as triangular or other shapes, thereby expanding the applicability of orthopedic fixation assemblies within these skeletal regions. Multiple varieties of trauma screws, including both lag screws-used to draw fracture segments together as exemplified in FIG. 1B—and standard fixation screws are represented, and scenarios such as those in FIG. 1C show the potential technical advantages of employing helicoid or piezoelectrically active screws in conjunction with trauma plates, increasing not only mechanical stability but providing osteogenic electrical stimulation as well. For the scenario in FIG. 1D, the orthopedic fixation assemblies are inserted into bone to stabilize spinal segments; however, in patient populations with osteoporotic or otherwise compromised bone quality, traditional screws may achieve limited purchase. In such environments, orthopedic fixation assemblies configured to induce local bone growth by delivering piezoelectrically generated signals around the screw can enhance the anchoring strength, improving spinal stabilization outcomes.

Further examples are demonstrated in FIGS. 1E and 1F. These figures present various forms of nails and pins, specifically as intramedullary orthopedic fixation assemblies 120E and 120F. The intramedullary orthopedic fixation assemblies shown embody smooth outer surfaces, though alternate surface finishes or modifications are also contemplated. FIG. 1E displays implant environments 100E, illustrating an intramedullary orthopedic fixation assembly in the form of a nail 120E traversing the length of a bone. The left portion of FIG. 1E features the device within a tibia, while the right portion presents implantation within a femur, in both cases within medullary cavities typically filled with bone marrow. In such scenarios, an intramedullary orthopedic fixation assembly, according to some embodiments, may be positioned at least partially within one or more bone cavities to stabilize fractures or support bone healing, while applying dynamic piezoelectric stimulation. FIG. 1F describes implant environments 100F, highlighting intramedullary orthopedic fixation assemblies as bone pins 120F spanning multiple bones in the foot, thus illustrating the diversity of anatomical use cases. In certain embodiments, these intramedullary orthopedic fixation assemblies may house one or more piezoelectric elements configured to generate and deliver electrical charge across the length of a bone, or across localized bone segments (such as regions adjacent to a fracture), thereby actively fostering osteogenesis and supporting enhanced healing and fixation.

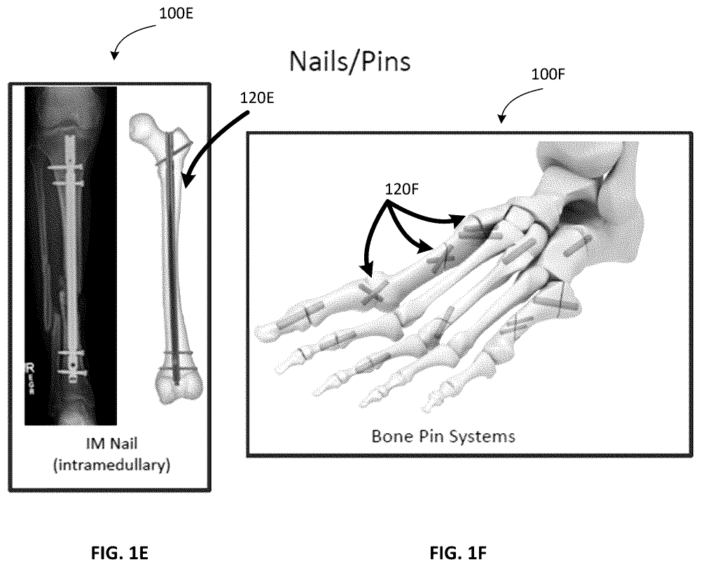

FIGS. 2A, 2B, and 2C illustrate various side elevational views of arthroplasty assemblies 200A, 200B, and 200C, each according to an embodiment. In arthroplasty implant assemblies 200A and 200B, the depicted constructs feature a femoral portion 201 comprising a femoral metal component 202, a polyethylene spacer 204, and a prosthetic stem 203. The tibial portion 205 includes its own polyethylene spacer 206, tibial components 208, and a tibial implant stem 207. The arrangement of these elements as shown permits restoration of knee joint mobility through mechanical articulation and resilient load distribution. Either the femoral stem 203 or the tibial stem 207, or both, may be configured according to some embodiments to possess inner surfaces oriented along directions that rotate with respect to one another as measured along the longitudinal axis of the orthopedic fixation assembly, such rotation supporting effective piezoelectric interfacial engagement and optimized transmission of dynamic anatomical forces across the device length as described elsewhere herein.

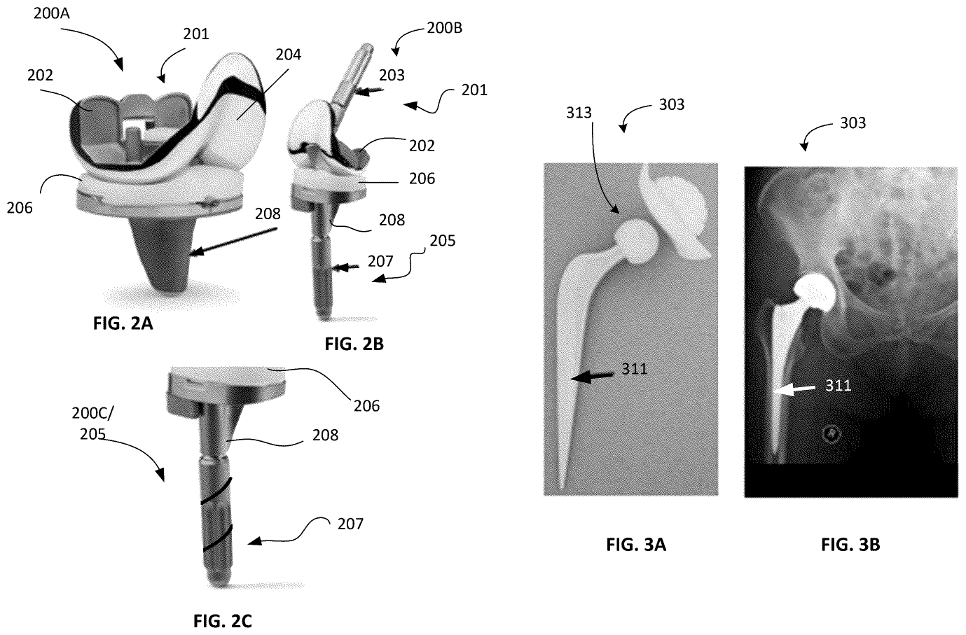

FIGS. 3A and 3B show x-ray images of a total hip prosthesis assembly 303 according to an embodiment. The prosthesis assembly 303 includes an articular head 313, which may be either a femoral head or a humeral head, and a prosthetic stem 311 that can be configured in accordance with the orthopedic fixation assembly of some embodiments. Prosthetic stem 311 is dimensioned and designed to fit within the intramedullary canal of either the femur (in hip replacement procedures) or the humerus (in shoulder replacements), where the stem supports the articular head and enables joint articulation and force transfer. This arrangement enables the device to mediate axial, bending, and rotational loads typical of physiologic hip or shoulder function, allowing for piezoelectric effects and mechanical stimulation of bone at the device interface. FIGS. 2A, 2B, and 2C present side elevational views of arthroplasty assemblies 200A, 200B, and 200C, each representing an embodiment. In assemblies 200A and 200B, the femoral portion 201 features a femoral metal component 202, a polymeric spacer 204, and a prosthetic stem 203. The tibial portion 205 is configured with a polyethylene spacer 206, tibial components 208, and a tibial implant stem 207. The illustrated assemblies are designed to restore knee joint mobility through effective load transfer and articulation, with the various spacers and components providing buffering and stability. In some embodiments, the femoral stem 203 and tibial stem 207 may be further configured so that their inner surfaces rotate in different directions along the longitudinal axis. This rotational arrangement distributes dynamic mechanical forces more effectively through the orthopedic fixation assembly across multiple contact planes, thereby enhancing both stability and the potential for piezoelectric stimulation when such features are present.

FIGS. 3A and 3B show x-ray images of a total hip prosthesis assembly 303, which includes an articular head 313 that may take the form of either a femoral head or humeral head. Prosthetic stem 311 may be configured as an orthopedic fixation assembly corresponding to some embodiments described herein. The stem 311 is dimensioned to fit within the intramedullary canal of the femur or humerus depending on the joint being replaced, providing subchondral support for the articular head while transmitting loads typical of physiological activity. These arrangements enable robust mechanical fixation, joint function, and, where utilized, enable piezoelectric stimulation to act on bone adjacent to the implant for improved osteogenesis.

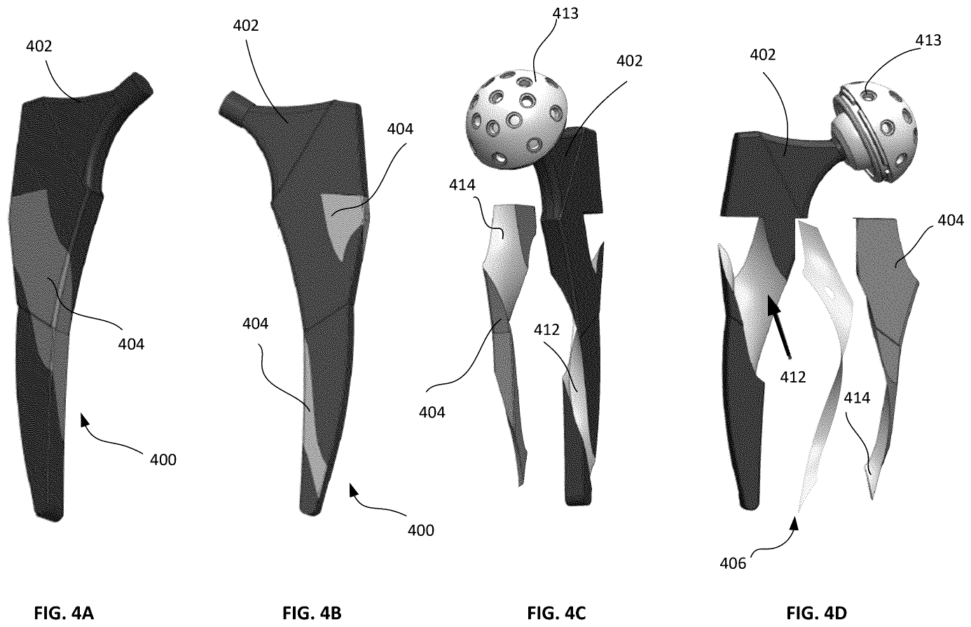

Referring to FIGS. 4A-4D, some embodiments provide an orthopedic fixation assembly 400 in the form of a prosthetic stem that is part of a total prosthesis assembly. In FIGS. 4C and 4D, the total prosthesis assembly is shown in exploded view and includes both orthopedic fixation assembly 400 and an articulated head 413, similar to the configuration illustrated in FIGS. 3A and 3B.

The structure of orthopedic fixation assembly 400 is made particularly clear in FIGS. 4A and 4B, where the assembly comprises a first lateral component 402 and a second lateral component 404. FIG. 4A provides a side perspective of one aspect of orthopedic fixation assembly 400, while FIG. 4B displays an opposing perspective, revealing the assembly's complete form from multiple angles. In the exploded views of FIGS. 4C and 4D, orthopedic fixation assembly 400 is again clearly depicted, along with the articulated head 413, and an internal piezoelectric material 406. The piezoelectric material 406 in this particular embodiment takes the form of a sheet interposed between first lateral component 402 and second lateral component 404, as further evidenced by the depictions in FIGS. 4A and 4B. The construction material for lateral components 402 and 404 is preferably an electrically conductive metal, such as titanium, allowing the entire structure to reliably transmit electrical effects generated by the piezoelectric component. Both lateral components are designed to engage each other in a helicoid configuration, where the first inner surface 412 of the first lateral component and the second inner surface 414 of the second lateral component twist about one another during assembly to create orthopedic fixation assembly 400. This helicoid engagement is not merely for mechanical joining, but serves the technical purpose of maximizing strain transferred to the piezoelectric material 406. Such a configuration efficiently strains the piezoelectric material in situ, producing electrical signaling that can stimulate osteogenesis in adjacent bone, while also mechanically securing the two components together with reduced need for additional fasteners, as the helicoid itself provides a self-interlocking feature.

In FIGS. 4C and 4D, the total prosthesis assembly is illustrated in an exploded view, presenting both orthopedic fixation assembly 400 and an articulated head 413, in a manner similar to the arrangement depicted in FIGS. 3A and 3B. orthopedic fixation assembly 400 is constructed of a first lateral component 402 and a second lateral component 404, as can be seen distinctly through the respective side perspective views of FIG. 4A and FIG. 4B. These figures provide comprehensive insight into the overall configuration from multiple orientations. FIGS. 4C and 4D also reveal the inclusion of a piezoelectric material 406 within the orthopedic fixation assembly 400, wherein the piezoelectric material is embodied as a sheet positioned between first lateral component 402 and second lateral component 404. These lateral components are preferably fabricated from an electrically conductive metal such as titanium, enhancing the transmission of piezoelectrically generated signals. In this embodiment, the first lateral component and the second lateral component are engineered to twist relative to each other during assembly, with the first inner surface 412 of the first lateral component and the second inner surface 414 of the second lateral component forming a helicoid configuration that wraps about the piezoelectric material 406. This helicoid engagement imparts significant technical advantages: it secures the piezoelectric material effectively, allows for efficient transfer of mechanical strain to the piezoelectric sheet resulting in high electrical output, and enables reliable mechanical union with reduced reliance on external fasteners due to the self-locking characteristic of the intertwining components.

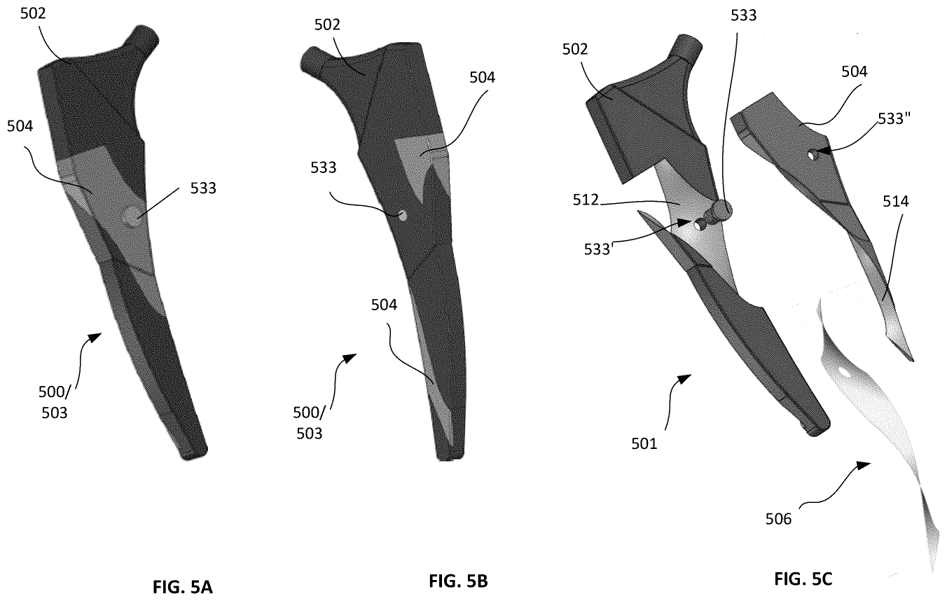

Turning to FIGS. 5A and 5B, orthopedic fixation assembly 500 has a construction that is identical to orthopedic fixation assembly 500, with the principal distinction being the inclusion of threaded holes within the lateral components 502 and 504 with respective first inner surface 512 and second inner surface 514, the later components forming the elongated body 503, and the addition of a screw 533 to anchor the components together. This aspect is further clarified in FIG. 5C, which illustrates a kit 501 comprising the first lateral component—defining a threaded hole 533′—and a second lateral component-defining a corresponding threaded hole 533″. During assembly, the respective threaded holes are brought into registration, and the screw 533 is inserted and rotated to solidly fix the two lateral components to each other. This supplementary mechanical fastening option, integrated with the helicoid feature, augments the structural integrity and adjustability of the assembly, supporting stability and securement while retaining all the piezoelectric and osteogenic technical benefits detailed above.

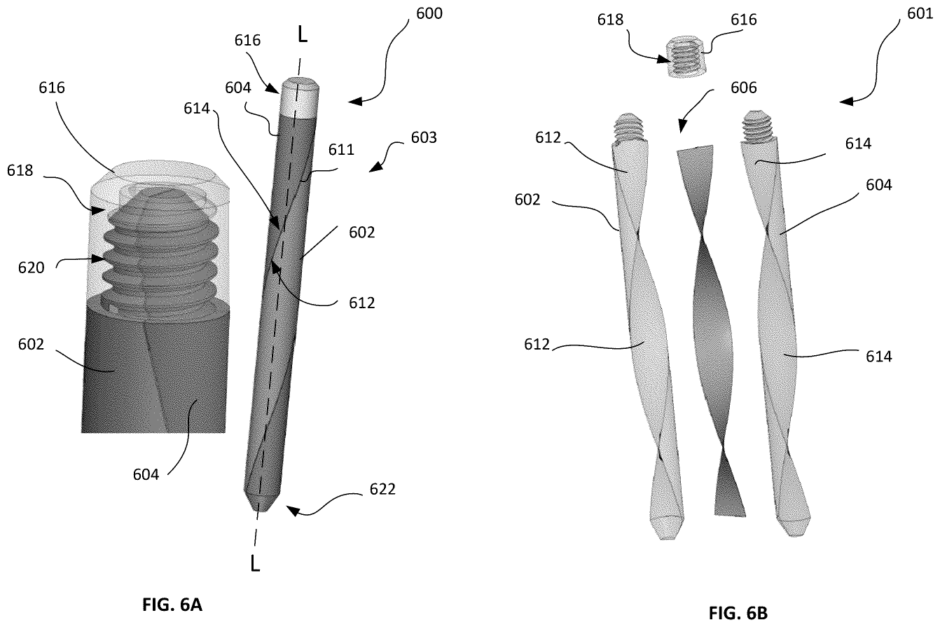

FIGS. 6A and 6B present a rod-shaped orthopedic fixation assembly 600 and an associated kit 601 for forming orthopedic fixation assembly 600.

As shown in FIG. 6A, orthopedic fixation assembly 600 comprises an elongated body 603 characterized by a non-threaded, smooth outer surface. The elongated body 603 includes a first lateral component 602 and a second lateral component 604, each having respective inner surfaces 612 and 614. These inner surfaces are positioned facing one another across an interface 611 that is clearly defined in FIG. 6A. The two lateral components 602 and 604 are designed to conform and mate together along the interface formed between their inner surfaces. Proximally, the elongated body 603 is equipped with a fastener cap 616, which in both FIGS. 6A and 6B is shown in a transparent representation to reveal its internal threading 618. Fastener cap 616 is adapted for engagement with a threaded head 620 of the elongated body 603; this threaded head 620 is formed when the first longitudinal component 602 and the second longitudinal component 604 are assembled as depicted in FIG. 6A. Distally, elongated body 603 includes a truncated cone 622, facilitating easier implantation into bone. The interface 611 is arranged for about 1.5 revolutions of the inner surfaces 612 and 614 relative to each other, providing secure, torsion-resistant assembly and promoting optimal transfer of mechanical loads.

The fastener cap may be made of an electrically insulating material, such as a polymeric material, such as, for example, PEEK or another similar material.

As shown in the exploded view of FIG. 6B, a piezoelectric material 606 of kit 601 is, in the final assembly 600, interposed between the two lateral components. Although the piezoelectric material 606, shown in the form of a sheet, is shown deformed as it would be in the final assembly, no embodiment herein is so limited. Embodiments include piezoelectric material in any shape, such as in a flat or undeformed shape which deforms in the final assembly, or one that is provided as deformed in a way so as to conform to the inner surfaces of the assembly's lateral components.

The embodiment illustrated by FIGS. 6A and 6B, is especially suitable for applications where rod-shaped orthopedic fixation assemblies are required, including clinical environments such as those shown in FIGS. 1E and 1F. The depicted embodiment demonstrates advantages in streamlined insertion, mechanical stability, and efficient piezoelectric energy transmission for promoting bone regeneration.

In some embodiments, the lateral components may self-lock through their helicoid engagement, either by the helicoid interface itself or by specialized mating features on the inner surfaces about the longitudinal axis L-L. The fastener cap 616 is further provided to prevent undesired longitudinal sliding between the joined components, assuring robust fixation during physiological loading and enhancing the overall structural integrity.

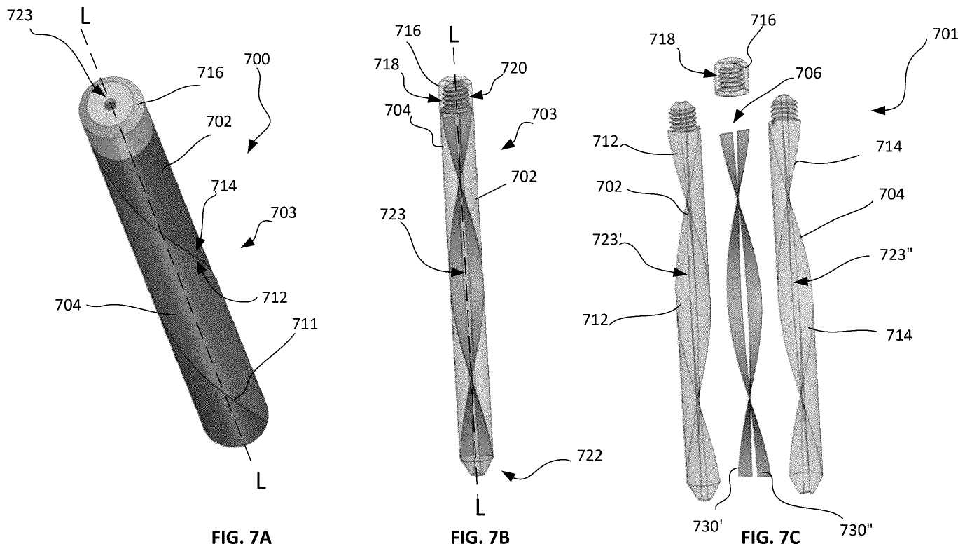

Turning now to FIGS. 7A-7C, another embodiment is provided in the form of orthopedic fixation assembly 700, which closely resembles orthopedic fixation assembly 600 but is further differentiated by the inclusion of a lumen 723 designed to accommodate a cannula that guides the implant during surgical placement. Referring to FIG. 7A, orthopedic fixation assembly 700 comprises an elongated body 703 with a non-threaded, smooth exterior. The structure of elongated body 703 is formed from a first lateral component 702 and a second lateral component 704, their respective inner surfaces 712 and 714 facing one another along a helicoid interface 711, well depicted in FIG. 7C. The lateral components 702 and 704 conform to one another in assembly, with the resulting structure capped proximally by fastener cap 716 with a threaded inner region 718 that mates with a threaded head 720 when the components are brought together. The distal portion terminates in a truncated cone 722 as seen in FIG. 7B, promoting ease of implantation. The helicoid interface 711 is designed to encompass approximately 1.5 revolutions of the inner surfaces, delivering exceptional interlocking and load transfer capabilities between the components. Self-locking mechanisms may be realized through the helicoid or other engagement methods at the interface, providing supplemental fixation beyond the fastener cap 716, which itself helps prevent unintended axial migration of the joined segments.

The fastener cap may be made of an electrically insulating material, such as a polymeric material, such as, for example, PEEK or another similar material.

A particular benefit of the configuration in FIGS. 7A-7C derives from the cannulated lumen 723 and its ability to allow for real-time positional guidance during implantation—a feature critical in applications requiring high placement precision, such as intramedullary or transosseous fixations.

Referring to FIG. 7C, kit 701 that, when assembled, forms orthopedic fixation assembly 700. Kit 701 includes an elongated piezoelectric material 706 configured for placement between the first and second lateral components 702 and 704. In some embodiments, piezoelectric material 706 resides directly at the helicoid interface 711, and may take either a pre-twisted conformal sheet shape as shown, or start as a flat sheet which adopts the twist geometry upon final assembly by virtue of being interposed between the lateral components. Kit 701 offers additional details, such as inclusion of two oblong piezoelectric components 730′ and 730″ (not visible in 7A and 7B) that span the helicoid interface but allow for uninterrupted lumen 723 registration-a feature created by corresponding grooves 723′ and 723″ defined in the respective inner surfaces of the lateral components. These registered grooves produce a continuous lumen 723 within the rod-shaped orthopedic fixation assembly 700 for guidewire passage, while the piezoelectric elements are arranged to maintain maximum energy transfer and signal output for bone stimulation.

The described system, as demonstrated by the cluster of FIGS. 7A-7C, exhibits technical advantages including enhanced precision of implant placement, robust self-locking helicoid architecture, secure axial fixation via fastener cap, and optimized piezoelectric stimulation across a segmented or continuous rod structure. These attributes collectively provide superior fixation, facilitate complex surgical procedures, and maximize biological healing responses, as can be further appreciated in rod-shaped orthopedic fixation assembly deployments within clinical use cases such as those shown in FIGS. 1E and 1F.

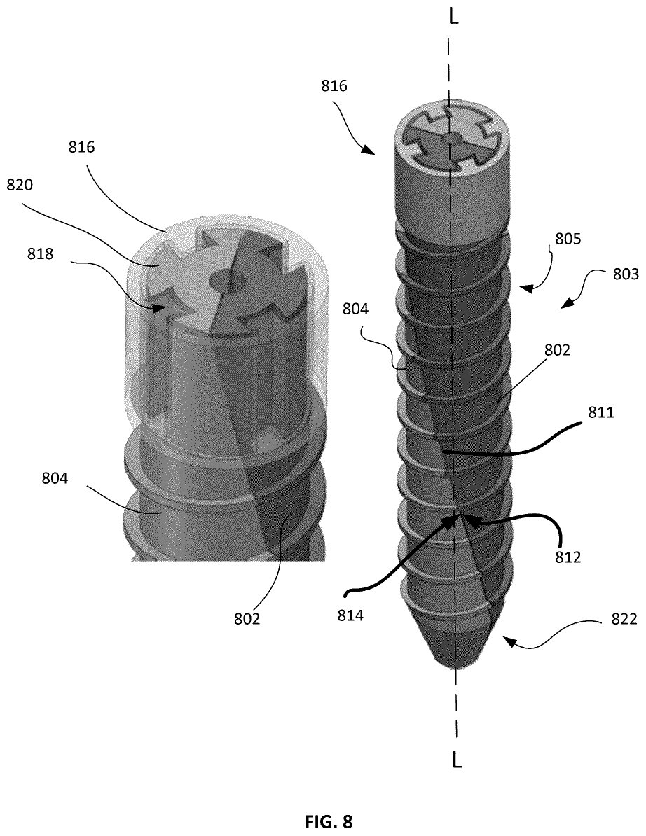

In FIG. 8, some embodiments provide an orthopedic fixation assembly 800 featuring an elongated body 803 with a threaded outer surface and threads 805. This elongated body 803 includes a first lateral component 802 and a second lateral component 804, each having inner surfaces 812 and 814, respectively, that face one another to define a helicoid interface 811. The two lateral components are configured to fit together in a conforming arrangement along the helicoid interface, yielding a robust, helicoid interlock.

At the proximal end of the elongated body 803, a fastener cap 816 is positioned. This fastener cap 816, as depicted in transparent view in FIG. 8, incorporates longitudinal splines 818 and wide grooves 819 that extend parallel to the longitudinal axis of the cap. The interior surface of the fastener cap is designed to mate with a splined head 820 of the elongated body, which is formed by assembling the first and second longitudinal components. The splines of the head 820 fit into the grooves of the cap, ensuring a strong press-fit connection that provides both rotational and axial stability.

The fastener cap may be made of an electrically insulating material, such as a polymeric material, such as, for example, PEEK or another similar material.

Distally, the elongated body 803 is shaped to include a truncated cone 822, which facilitates easier insertion and guidance during surgical implantation. The helicoid interface 811 between the lateral components in this embodiment encompasses approximately 0.8 revolution about the longitudinal axis L-L, allowing the helicoid arrangement to provide self-locking engagement that further enhances fixation integrity.

The fastener cap 816 serves not only to anchor the proximal ends of the lateral components but also to prevent unwanted longitudinal sliding, further reinforcing the assembly during physiological loading.

This embodiment, as illustrated in FIG. 8, may be ideally suited for applications requiring threaded orthopedic fixation assemblies or orthopedic screws, such as those depicted in FIGS. 1A-1D. While this particular embodiment demonstrates an orthopedic fixation assembly shaped like a screw with an internally splined fastener cap, other fastener designs and orthopedic fixation assembly body shapes may be realized in additional embodiments, offering flexibility to address varying clinical and anatomical requirements.

According to some embodiments, a fastener cap may be constructed from either an electrically insulating material or an electrically conductive material. In situations where a conductive material is selected, an electrically insulating liner, such as a liner formed from PEEK, may be disposed between the elongated body and the fastener cap to maintain necessary electrical isolation.

Although FIGS. 6A-6B, 7A-7C and 8 depict fastener caps whose outer surfaces are substantially flush with the outermost surface of the elongated body, these embodiments are not limited to this configuration. Fastener caps may alternatively be designed with outer surfaces that are recessed relative to the outermost surface of the elongated body, or with outer surfaces that protrude outward beyond the outermost surface.

In addition, despite FIGS. 6A-6B, 7A-7C and 8 illustrate fastener caps at only one end of the depicted orthopedic fixation assemblies, further embodiments may provide a fastener cap at just one or at both the proximal and distal ends of the elongated body. This flexibility allows adaptation of the assembly to varying clinical and structural requirements.

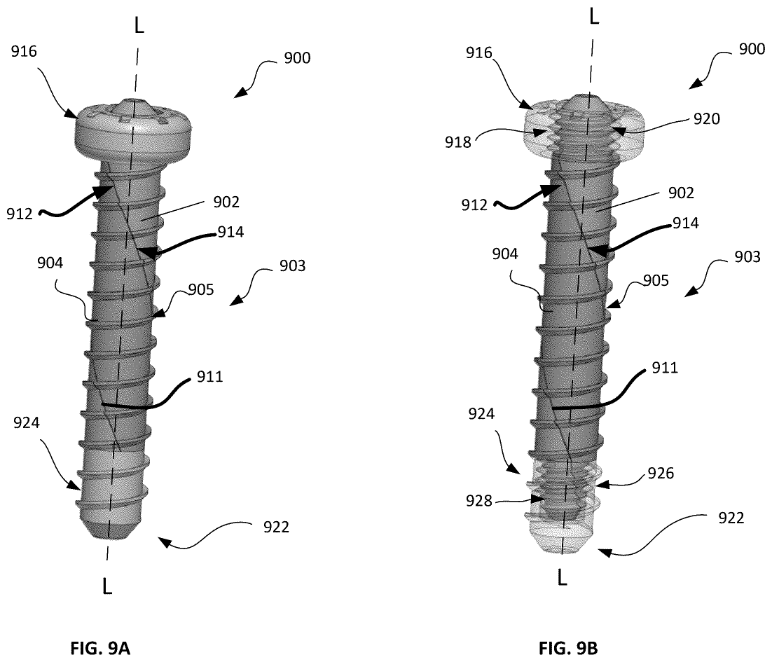

In FIGS. 9A and 9B, some embodiments provide an orthopedic fixation assembly 900 that comprises an elongated body 903 having a threaded outer surface with threads 905. The elongated body 903 includes a first lateral component 902 and a second lateral component 904, each with respective inner surfaces 912 and 914. These inner surfaces face each other and define a helicoid interface 911, allowing the two lateral components to conform and interlock securely upon assembly.

At the proximal end of elongated body 903 is a fastener cap 916, while a second fastener cap 924 is positioned at the distal end. FIG. 9B depicts these fastener caps 916 and 924 in transparent form to reveal their threaded interiors 918 and 926, respectively. Each threaded interior is designed to engage with a corresponding threaded head or tip of the elongated body: threaded head 920 at the proximal end and threaded tip 928 at the distal end. These threads are formed by the mating of the first and second longitudinal components 902 and 904 during assembly. In some embodiments, the distal fastener cap 924 has a truncated cone 922 to facilitate ease of implantation.

The fastener cap may be made of an electrically insulating material, such as a polymeric material, such as, for example, PEEK or another similar material.

The fastener caps in this embodiment can be configured with an outwardly protruding, or “positive,” profile relative to the outermost surface of the elongated body 903, such that they define a distinct “head” structure for the orthopedic fixation assembly 900. The interface 911 extends for approximately 0.8 revolution of the relative inner surfaces, enabling self-locking engagement as the components are assembled about the longitudinal axis. The helicoid nature of the interface helps immobilize the lateral components and enhances load transfer, while both fastener caps 916 and 924 prevent longitudinal sliding between the components and further strengthen fixation during dynamic physiological loading.

This embodiment, as depicted in FIGS. 9A and 9B, is appropriate for use in any clinical scenario requiring a threaded orthopedic fixation assembly or orthopedic screw, including scenarios like those seen in FIGS. 1A through 1D. For those embodiments in which the elongated body has a threaded shaft, the thread may extend along the entire length or only part of the shaft, such that some segments remain threaded while others are smooth. Examples of such partially or fully threaded orthopedic fixation assemblies include lag screws, as shown in FIG. 1B, where differential threading and unthreaded regions support varied fracture fixation and compression strategies in orthopedic practice.

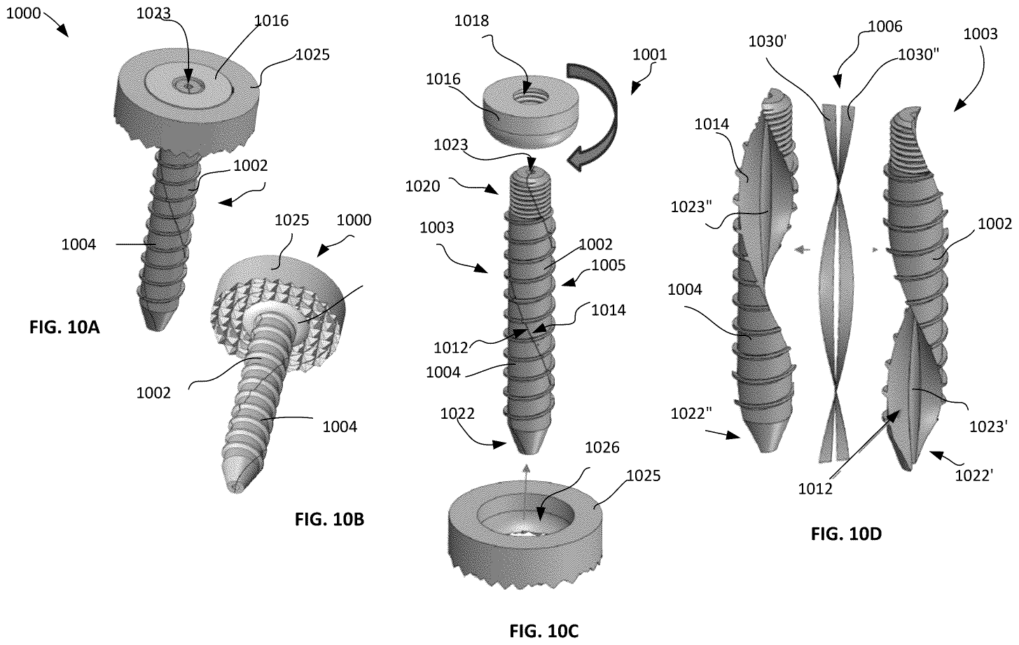

FIGS. 10A-10C illustrate another embodiment of an orthopedic fixation assembly 1000 designed in the form of a screw, while FIG. 10D provides an exploded view of the elongated body of orthopedic fixation assembly 1000 shown in FIGS. 10A-10C.

FIG. 10A presents a top perspective of orthopedic fixation assembly 1000, FIG. 10B offers a bottom perspective, and FIG. 10C shows a top perspective of a kit 1001 configured so that, once assembled, it forms orthopedic fixation assembly 1000.

Orthopedic fixation assembly 1000 features an elongated body 1003 having a threaded outer surface defined by threads 1005. The assembly includes a first lateral component 1002 and a second lateral component 1004, with each component providing respective inner surfaces 1012 and 1014. These inner surfaces face each other and together establish an interface 1011. The two lateral components 1002 and 1004 are designed to conform to one another at this interface, ensuring secure mating along the helicoid.

At the proximal end, elongated body 1003 incorporates a fastener cap 1016. The inner surface 1018 of this fastener cap is threaded and engages a threaded head 1020 of the elongated body that is created by the assembly of components 1002 and 1004. The assembly further includes a washer portion 1025 featuring a recessed inner region 1026 to receive the fastener cap 1016. As best seen in FIG. 10B, washer portion 1025 may incorporate protrusions, such as teeth, which extend from its bottom surface to assist with integration into bone.

The fastener cap may be made of an electrically insulating material, such as a polymeric material, such as, for example, PEEK or another similar material. The fastener cap may further be made of an electrically conductive material (by virtue of the presence of washer portion 1025), such as, for example, tungsten and/or other metals.

The distal end of elongated body 1003 features a truncated cone 1022, aiding the ease of insertion during surgical implantation. The helicoid interface 1011 of this embodiment is arranged for about 1.5 revolutions of the inner surfaces, further augmenting self-locking characteristics by providing increased rotational and axial engagement.

The fastener cap 1016 helps prevent undesired longitudinal movement of the joined lateral components and promotes stable fixation. Referring specifically to FIG. 10D, the exploded view highlights the first lateral component 1002, the second lateral component 1004, and a piezoelectric material 1006 that is positioned to be interposed between them. The piezoelectric material may include two oblong piezoelectric components 1030′ and 1030″ (not visible in FIGS. 10A-10C), and the helicoid of inner surfaces 1012 and 1014 is clearly visible.

As further illustrated in FIG. 10D, the first lateral component 1002 defines a longitudinal groove 1023′ at inner surface 1012, while the second lateral component 1004 defines a longitudinal groove 1023″ at inner surface 1014. In assembly, grooves 1023′ and 1023″ are brought into registration to form a lumen 1023 extending along the length of orthopedic fixation assembly 1000, which can be used for insertion of a guidewire to direct the assembly to the target bone site. This configuration enables the piezoelectric material to be arranged so as to maximize available lumen space for such a guidewire, while still supporting electrical functionality.

Such embodiments, as seen in FIGS. 10A-10D, are well suited for use wherever threaded orthopedic fixation assemblies or orthopedic fixation assembly screws are indicated, as in the surgical environments represented by FIGS. 2A-2C. While these particular figures focus on a screw-shaped orthopedic fixation assembly with a threaded fastener cap design, further variations can include other fastener component geometries and orthopedic fixation assembly shapes as called for to address a range of anatomical or surgical requirements.

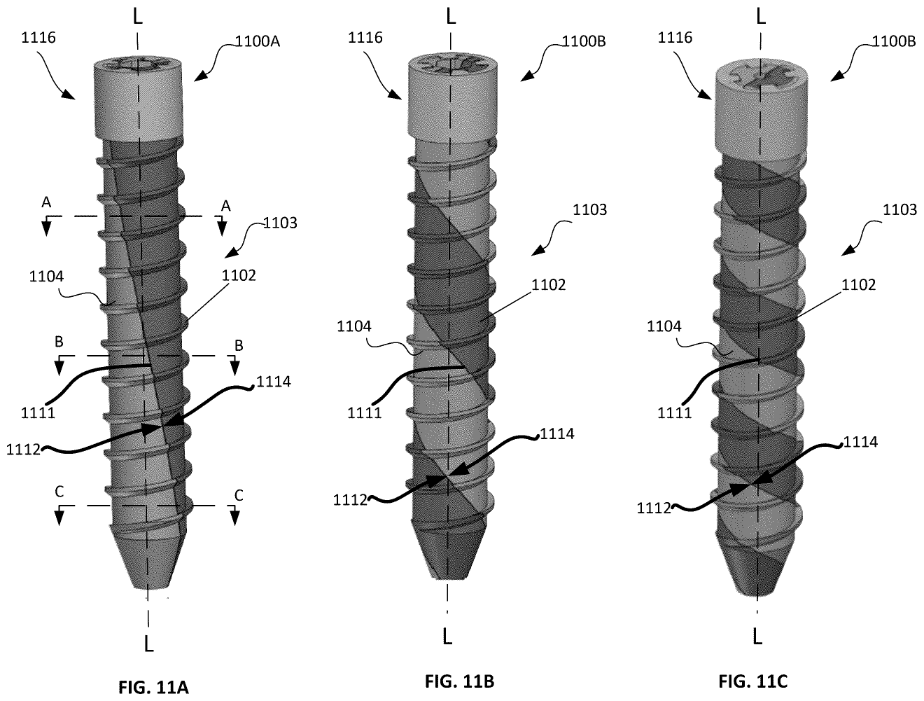

FIGS. 11A-11C display orthopedic fixation assemblies 1100A, 1100B, and 1100C, each corresponding to screw-type devices. These orthopedic fixation assemblies share general correspondence with the configuration of orthopedic fixation assembly 800 shown in FIG. 8, and for reference, like components between FIGS. 11A-11C and FIG. 8 are denoted with like reference numerals. For example, orthopedic fixation assembly 1100A represents the configuration of orthopedic fixation assembly 800, but with the helicoid interface 1111 spanning approximately 0.8 revolution along the longitudinal axis L-L. orthopedic fixation assembly 1100B similarly tracks the arrangement of orthopedic fixation assembly 800 but is distinguished by having two complete revolutions for its interface 1111, also oriented along the longitudinal axis L-L.

Within FIGS. 11A-11C, each orthopedic fixation assembly incorporates an elongated body 1103 with a threaded exterior. The assembly includes a first lateral component 1102 and a second lateral component 1104, with each component defining inner surfaces 1112 and 1114 that are joined along the helicoid interface 1111. The elongated body 1103 is provided at its proximal end with a fastener cap 1116. In the sets of embodiments shown, the helicoid interface 1111 is varied to provide different engagement lengths and mechanical properties: in orthopedic fixation assembly 1100A about 0.8 revolution, in orthopedic fixation assembly 1100B two revolutions, and in orthopedic fixation assembly 1100C four revolutions, all rotations being measured about the device's longitudinal axis L-L.

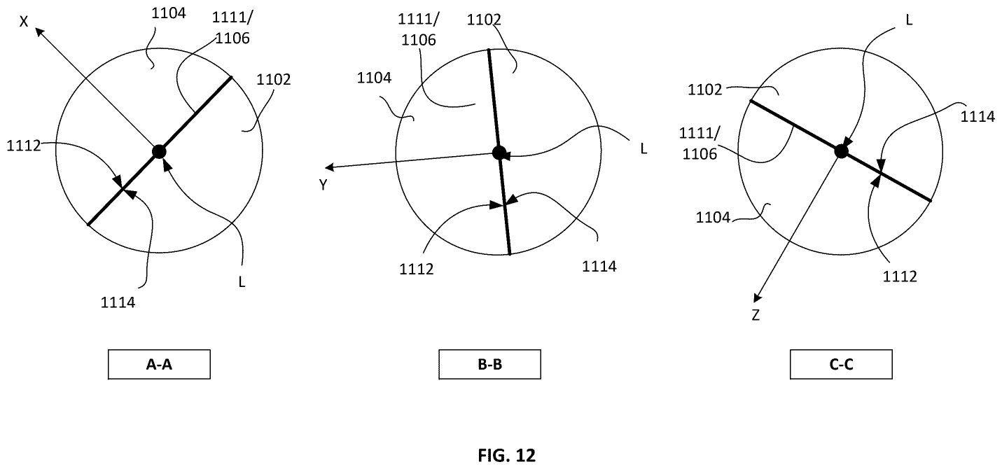

FIG. 12 provides top plan views of the elongated body 1103 (as from orthopedic fixation assembly 1100A), showing schematic cross-sectional slices at positions A, B, and C along the device, without showing threaded portions for the sake of simplicity. In each of these views, first lateral component 1102 can be seen interfacing with second lateral component 1104, with the piezoelectric material 1106 clearly interposed between the two lateral components. The orientation of these cross-sections is along the longitudinal axis L-L, and the aligned directions X, Y, and Z are as indicated in the figures. X, Y and Z rotate about longitudinal axis L-L as we move down the elongated body, signaling the existence of the helicoid interface.

By virtue of the spiral or helicoid configuration of interface 1111, as can be readily seen from FIG. 12, at a location A along the elongated body 1103 of FIG. 11A as seen in top plan view in the direction of L-L, a normal vector NVA orthogonal to the first inner surface 1112 is in a direction X; and at a location B below location A along the elongated body 1103, a normal vector NVB to the first inner surface 1112 is in a direction Y, wherein, as seen in FIG. 12, direction Y is rotated about the longitudinal axis of the elongated body as compared to direction X, because of the spiraling of the inner surfaces of the lateral components of the elongated body with respect to one another.

According to some embodiments, an orthopedic fixation assembly includes a location A and a location B along its length, where the direction Y at location B is rotated by between about 90 degrees and about 360 degrees relative to direction X at location A. For multiple revolutions of the interface between the inner surfaces, some embodiments, according to a second aspect, at a third location C below location B along the elongated body 1103, a normal vector NVC to the first inner surface extends in a direction Z.

According to some embodiments, an orthopedic fixation assembly includes not only: (1) a location A and a location B along its length, where the direction Y at location B is rotated by between about 90 degrees and about 360 degrees relative to direction X at location A, but also (2) a location B and a location C along its length, where the direction Z at location C is rotated by between about 90 degrees and about 360 degrees relative to direction X at location B. The latter description is to take into account spiraling that is partial (e.g., ¼ revolution or a 90 degree differential between directions X and Y), up to at least 2 revolutions or a 360 degrees+360 degrees rotation of the first inner surface as between locations A and B.

According to some embodiments, an orthopedic fixation assembly may define multiple revolutions, such that, in general, at a location N below an immediately previous location N-1 along the elongated body, a normal vector NVN to the first inner surface extends in a direction I, wherein I is rotated about the longitudinal axis of the elongated body by an angle between about 90 degrees and about 360 degrees relative to the direction of a normal vector NVN-1 at location N-1.

Although the above description is given in the context of the first inner surface, it goes without saying that the same description would be applicable to the second inner surface. The description is essentially to highlight a spiraling of the inner surfaces about the longitudinal axis of an orthopedic fixation assembly according to some embodiments. According to some embodiments, a number of revolutions is between about 0.8 revolution and about 4 revolutions, as depicted by way of some of the examples given in FIGS. 11A-11C.

Although FIG. 12 illustrates the interface between the lateral components as being substantially flat in the depicted cross-sections at locations A, B, and C, it is to be understood that the interface is not limited to a linear configuration. In alternative embodiments, the interface in such cross-sectional views may assume any suitable shape, including but not limited to non-planar geometries or profiles. For example, the interface may exhibit one or more undulations, such as wavelike or sinusoidal profiles, or may include zig-zag patterns. Additional non-planar shapes may include sawtooth profiles, stepped or terraced configurations, corrugated surfaces, or combinations thereof. The interface may also be irregular, asymmetric, or otherwise contoured to enhance mechanical interlocking, increase surface area, or optimize strain transfer to the piezoelectric material.

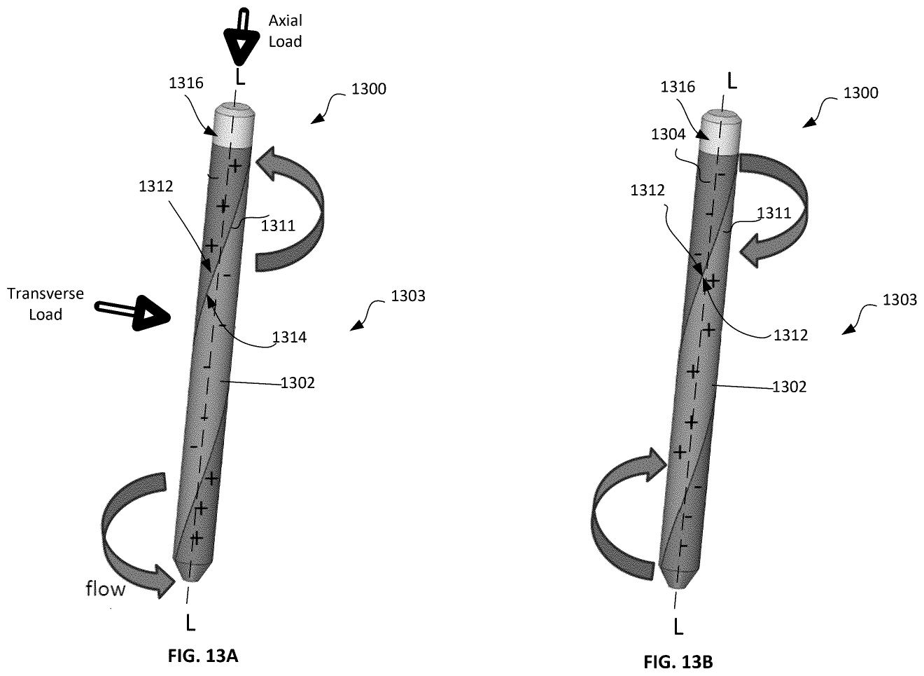

FIGS. 13A and 13B provide illustrative representations of the orthopedic fixation assembly 1300 of FIG. 6A, highlighting the behavior of the device under both loaded and unloaded conditions. In FIG. 13A, the orthopedic fixation assembly 1300 is depicted while subject to an axial and/or transverse load. Under such loading, the first lateral component 1302 is induced to assume one electrical polarity by virtue of the piezoelectric effect generated by the piezoelectric material incorporated within the assembly, while the second lateral component 1304 assumes an opposite polarity. In the example shown, the first lateral component 1302 develops a positive charge, whereas the second lateral component 1304 develops a negative charge.

As a result, electron flow within the orthopedic fixation assembly 1300, as visually indicated in FIG. 13A, proceeds from the second lateral component 1304 toward the first lateral component 1302. Due to the spiral or helicoid configuration of the lateral components, this current pathway can adopt varying spatial directions along the length of the orthopedic fixation assembly 1300, producing a localized distribution of electrical stimulus. Such a distribution is advantageous in promoting bone growth in targeted regions adjacent to the implant, thereby supporting enhanced osteogenesis and fixation.

FIG. 13B illustrates the electrical response of the orthopedic fixation assembly 1300 after the applied load has been released. In this scenario, the first lateral component 1302 now exhibits a charge polarity opposite to that observed under load, and likewise for the second lateral component 1304. Specifically, the first lateral component 1302 becomes negatively charged, and the second lateral component 1304 becomes positively charged. Consequently, electron flow is reversed, proceeding from the first lateral component 1302 toward the second lateral component 1304. The spiral or helicoid structure again results in spatially distinct current pathways that are oriented differently along the longitudinal extent of the device relative to the loaded state. This dynamic reversal of current and charge polarity with each loading cycle serves to differentially stimulate bone tissue in the vicinity of the orthopedic fixation assembly 1300 and is anticipated to further enhance bone healing and long-term integration.

In some embodiments, the orthopedic fixation assembly is configured such that cyclic loading and unloading during physiological use generates an alternating current (AC) effect within the piezoelectric material. An example of such an environment is shown in FIGS. 13A and 13B. As the implant is subjected to mechanical loads, the piezoelectric material produces an electrical charge of a first polarity, and upon release of the load, the polarity reverses. This dynamic polarity reversal results in an alternating electrical signal at the implant site, which more closely mimics the natural electrical environment of bone. The alternating current effect is believed to enhance the recruitment and activity of bone-forming cells, thereby promoting more robust and rapid bone healing and integration.

The design of the orthopedic fixation assembly in some embodiments is intended to biomimetically replicate the natural piezoelectric properties of bone tissue. Natural bone generates electrical signals in response to mechanical stress, which are thought to play a key role in signaling bone growth and remodeling. By incorporating a piezoelectric material at the interface of the lateral components, and by configuring the assembly to generate electrical signals under physiological loading, the device is able to provide a biomimetic stimulus that encourages osteogenesis, which is particularly beneficial in patients with compromised bone quality or in cases where cementless fixation is desired.

In certain embodiments, the orthopedic fixation assembly may be manufactured using additive manufacturing such as 3D printing or subtractive techniques such as wire electrical discharge machining. The method of manufacture can impart distinct surface characteristics to the components, such as surface roughness, striations, or other textural features. These manufacturing-induced features may provide functional benefits, such as improved bone ongrowth or enhanced mechanical interlocking between components. For example, a 3D-printed component may exhibit a characteristic layer-by-layer texture, while a wire EDM component may have a smoother, machined finish. The presence of such features can be used to identify the manufacturing method and may be claimed as a structural limitation.

In some embodiments, the piezoelectric material is provided in a discontinuous or segmented form, such as multiple strips, segments, or discrete elements, rather than as a single continuous sheet. This configuration facilitates assembly, particularly in cases where the piezoelectric material is relatively thick or where a central lumen is required for a guidewire. By segmenting the piezoelectric material, it is possible to maintain an unobstructed lumen while still providing effective piezoelectric stimulation at the interface. Segmented piezoelectric elements may reduce the risk of material wrinkling, folding, or damage during assembly, and can be strategically positioned to optimize electrical output in regions of highest mechanical stress.

The number of helical or helicoid revolutions defined by the interface between the lateral components may be selected based on the intended clinical application and the expected loading environment. For example, in applications where the implant is subject to significant bending loads, such as pedicle screw rods, a greater number of revolutions may be used to enhance self-interlocking and mechanical stability. In contrast, for applications primarily subject to compressive loads, such as sacroiliac joint screws, fewer revolutions may be sufficient. This design flexibility allows the orthopedic fixation assembly to be tailored to specific anatomical sites and loading conditions, optimizing both mechanical performance and ease of assembly.

The self-interlocking property of the orthopedic fixation assembly is enhanced as the number of helical revolutions increases. When the interface between the lateral components wraps around the longitudinal axis by more than one full revolution, the components become increasingly resistant to separation in directions perpendicular to the axis. This self-interlocking effect reduces the need for additional fasteners and improves the overall stability of the assembly, particularly under torsional or bending loads. The ability to achieve robust fixation through geometric interlocking is a significant advantage in minimizing hardware complexity and potential points of failure.

In some embodiments, the orthopedic fixation assembly may be provided as a modular kit or as a blank helical construct that can be further machined or finished for specific applications. For example, a helical blank may be manufactured with a generic outer profile and then customized with threads, surface features, or attachment points to suit a particular surgical indication. This modular approach allows for intraoperative customization, inventory reduction, and compatibility with a range of fasteners or heads. The kit may include interchangeable components, such as different fastener caps or washers, enabling the surgeon to adapt the assembly to the patient's anatomy and the clinical scenario.

The orthopedic fixation assembly may be designed for compatibility with existing orthopedic systems and instrumentation. For example, the helical assembly may be dimensioned to fit standard pedicle screw tulips, trauma plates, or other fixation hardware commonly used in orthopedic surgery. This compatibility allows the novel piezoelectric assembly to be integrated into established surgical workflows and to leverage existing tools and implants, facilitating adoption and reducing the learning curve for surgeons.

In addition to manufacturing-induced surface features, the outer surfaces of the lateral components may be intentionally roughened, textured, or made porous to promote bone ongrowth and osseointegration. Porous or roughened surfaces can enhance the biological fixation of the implant by providing a scaffold for bone ingrowth, improving long-term stability. The specific surface characteristics may be selected based on the intended application, the method of manufacture, and the desired biological response.

The orthopedic fixation assembly may utilize either press fit or threaded fastener caps to secure the lateral components. Threaded caps allow for controlled torque application and robust fixation, which is particularly advantageous in applications subject to high mechanical loads. Press fit caps provide a simpler and potentially quicker method of assembly, relying on friction and surface area for retention. The choice between press fit and threaded caps can be made based on the clinical scenario, desired ease of use, and required fixation strength.

In some embodiments, the assembly may include a washer portion with protrusions, such as teeth or spikes, configured to engage the surrounding bone. This feature distributes the load over a larger surface area, reduces the risk of implant subsidence, and enhances the initial mechanical stability of the fixation construct. The washer may be integrated with the fastener cap or provided as a separate component in the kit.

For applications requiring a central lumen, such as for guidewire placement, the piezoelectric material may be provided in a split or bifurcated configuration. In this design, the piezoelectric elements are positioned on either side of the lumen, maintaining electrical functionality while preserving an unobstructed passage for the guidewire. This approach is particularly useful in minimally invasive procedures or in implants that must be delivered over a guidewire.

The pitch of the helical interface between the lateral components may be varied along the length of the implant. For example, a higher pitch at one end may facilitate easier assembly or insertion, while a lower pitch at the other end may enhance mechanical interlocking and load transfer. Variable pitch designs allow the implant to be optimized for both surgical handling and in vivo performance, providing a balance between ease of use and fixation strength.

The provision of a spiral, or helicoid, interface incorporating an electromechanical piezoelectric material (EMP) presents significant advancements in the field of orthopedic devices. This configuration is particularly applicable to elongated implants such as screws, nails, pins, and arthroplasty stems. The helicoid interface with embedded EMP imparts a range of technical and clinical benefits that address longstanding challenges in bone fixation, healing, and integration. The helicoid interface with EMP enables efficient transfer of mechanical strain to the EMP, thereby actuating the piezoelectric effect under dynamic loading. It provides biomimetic electrical stimulation that promotes bone growth and healing, allows for increased EMP volume within the implant resulting in enhanced charge generation, supports modularity and versatility in design permitting adaptation to a variety of orthopedic applications, affords manufacturing and design flexibility accommodating different fabrication methods and design features, and facilitates broad patent coverage and design protection through its adaptable structure and range of embodiments.

The helicoid configuration is particularly effective in transmitting mechanical loads, including compression, bending, and torsion, from the implant structure to the embedded EMP. By situating the EMP between helicoid fractions, the device ensures that dynamic loading from any anatomical direction is efficiently conveyed to the piezoelectric material. This feature is especially advantageous in anatomical sites where loading is multidirectional and variable, such as in hip stems and spinal rods. The result is reliable actuation of the piezoelectric effect, regardless of the orientation or nature of the applied force.

Natural bone tissue exhibits inherent piezoelectric properties, generating electrical signals in response to mechanical stress. These signals play a critical role in stimulating bone growth and remodeling. The helicoid interface with embedded EMP is designed to mimic this natural process. As the implant is loaded and unloaded during normal patient movement, the EMP produces alternating electrical signals. This alternating current effect is believed to enhance the recruitment of bone-forming cells and improve osseointegration, which is particularly beneficial for patients with compromised bone quality, such as those with osteoporosis, or in cases where cementless fixation is employed.

The helicoid configuration permits the incorporation of a greater length of EMP material within the implant, especially as the number of helicoid revolutions increases. This expanded volume of EMP enables the device to generate higher electrical charges under mechanical loading conditions. The increased charge generation is expected to improve the clinical efficacy of the implant in stimulating bone growth and accelerating healing at the site of implantation.

The helicoid interface is inherently modular and versatile, allowing for adaptation to a wide range of orthopedic applications. The number of helicoid revolutions, the pitch, and the presence or absence of threads can be tailored to meet the specific mechanical and anatomical requirements of each use case. For example, constructs requiring greater bending strength, such as pedicle screw rods, may utilize a higher number of revolutions, while applications subject primarily to compressive loads, such as SI joint screws, may employ fewer revolutions. This adaptability supports the use of the helicoid interface in diverse clinical scenarios.

Through experimentation, the inventors have discovered that, in the context of the orthopedic fixation assemblies described herein, the configuration of the helicoid interface between the first and second lateral components can have a significant impact on the electrical output generated by the piezoelectric material under physiological loading. When a piezoelectric material is positioned at an interface defined by a helicoid shape—such as a spiral or helical configuration about a line extending through the elongated body—the application of a given axial, compressive, shear, or combined force to the assembly results in a higher voltage output across the piezoelectric material compared to assemblies with a flat or non-helicoid interface.

The inventors have further observed that increasing the number of helicoid revolutions, or varying the pitch of the helicoid interface, can further enhance the electrical output generated by the piezoelectric material. This is believed to be due to the more efficient transfer and distribution of mechanical forces along the helicoid path, resulting in greater deformation of the piezoelectric material and, consequently, increased electrical stimulation.

These increased voltage outputs are advantageous, as they are associated with improved osteogenesis, including faster and more robust bone growth at the implantation site. For example, pre-clinical research by the inventors has demonstrated that orthopedic fixation assemblies incorporating a helicoid interface with multiple revolutions result in significantly enhanced bone formation compared to assemblies with a flat or planar interface. This effect is attributed to the unique mechanical environment created by the helicoid geometry, which promotes more effective activation of the piezoelectric material under physiological loading.

Accordingly, in some embodiments, the orthopedic fixation assembly is configured such that the interface between the first and second lateral components, and the piezoelectric material positioned therebetween, defines a helicoid shape about a line extending through the elongated body. This configuration enables the device to provide superior electrical stimulation under physiological loading, thereby promoting improved bone healing and integration without the need for external power sources, batteries, wires, or circuits. These findings support the use of helicoid interfaces in orthopedic fixation assemblies for enhanced clinical outcomes.

The helicoid interface can be fabricated using various manufacturing techniques, including additive methods such as 3D printing and subtractive methods such as wire electrical discharge machining. The design accommodates discontinuous or segmented placement of the EMP, variable pitch, and the inclusion of undulating or textured surfaces to further enhance bone integration or mechanical performance. The modular nature of the design also allows for the use of interchangeable fasteners and compatibility with existing orthopedic systems, such as pedicle screw tulips and trauma plates.

In some of the shown embodiments, although rods are depicted as having a cylindrical cross section, embodiments are not so limited, and include within their scope the provision of rods of any cross section, such as oval, polygonal, rectangular, square, star-shaped, etc.

Although embodiments as described herein show a dual lateral component orthopedic fixation assembly, embodiments are not so limited, and include within their scope any number of lateral components which together define an interface to impart a helicoid effect to charge generation by a piezoelectric material at the interface.

As used herein, the phrase “a line extending through the elongated body” is not limited to a particular geometry or position within the assembly. The line may be straight or curved, and may correspond to a midline, a longitudinal axis, or any other axis extending through the elongated body. In some embodiments, the line may be centrally located, while in other embodiments it may be offset or follow a non-linear path, depending on the desired configuration of the interface or the anatomical requirements of the application.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “comprising” and “comprises of” includes the more restrictive claims such as “consisting essentially of” and “consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out embodiments described herein.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

As used herein, “electrically conductive” in some examples may refer to a property of a material having an electrical conductivity greater than or equal to 106 Siemens per meter (S/m) at 20 degrees Celsius. Examples of such materials include titanium (with relatively low electrical conductivity compared to many other metals, that is, about 2.4×106 S/m) and tungsten (with much higher electrical conductivity of about 1.8×107 S/m).

As used herein, an “electrically insulating material” may include a polymer, such as, for example, PEEK (Polyether Ether Ketone), Polyimide (PI), Polyphenylene Sulfide (PPS), Polyaryletherketone (PAEK), Polyetherketone (PEK), Polyetherketoneketone (PEKK), and/or Fluoropolymers.

As used herein, holes that “register” or that are in “registration” with one another are holes that are either in full registration (holes that overlap one another fully) with one another, or in partial registration with one another (holes that overlap one another partially).

Holes as described herein may be through holes or blind holes. Such holes may be provided, for example, by way of machining, or using any other suitable technique.

Lateral components, fasteners such as screws, pins and/or caps as described herein may be unitary (i.e., a one piece component) or they may have multiple attached components that together form such components. Such components may be formed, for example, using 3D printing, or any other suitable technique.

Any component described herein as being made of an electrically conductive material may be formed from or include any one or more of titanium, stainless steel (such as 316L surgical stainless steel), cobalt-chromium alloys (such as CoCrMo), tantalum, niobium, gold, platinum, silver, tungsten, conductive medical-grade polymers or composites (such as carbon fiber-reinforced polymers), or other biocompatible metal alloys. The selection of material may include any combination of these materials, and may be based on factors such as electrical conductivity, biocompatibility, corrosion resistance, and mechanical properties suitable for orthopedic or implantable medical devices.

According to some embodiments, a piezoelectric material may be formed of any one or more of polyvinylidene fluoride (PVDF), polyvinylidene fluoride-trifluoroethylene (PVDF-TrFE), polyether ether ketone (PEEK), piezoelectric single-crystal fibers or whiskers, or conductive medical-grade polymers or composites, such as carbon fiber-reinforced polymers, either alone or as a matrix including piezoelectric fillers. These materials may in some embodiments be selected for their ability to bend and flex without breaking, making them particularly suitable for applications where the piezoelectric component must conform to curved or helicoid interfaces or withstand repeated physiological loading. Rigid piezoelectric ceramics such as lead zirconate titanate (PZT), barium titanate, and piezoelectric ceramic fibers, rods, or particles, according to some embodiments, may be incorporated as fillers within a flexible polymer matrix. Accordingly, the piezoelectric material may include any combination of PVDF, PVDF-TrFE, PEEK, flexible single-crystal fibers or whiskers, and flexible piezoelectric polymer composites, with or without embedded piezoelectric fillers, to achieve the desired balance of piezoelectric performance, flexibility, and biocompatibility for orthopedic or implantable medical devices.

Any one of the piezoelectric layers as described herein may be substantially planar in shape prior to assembly, and deformed into a non-planar shape, such as having undulations, waves, zig-zags, curves, creases, and/or bends, due to the application of a force to the top and bottom lateral components. The piezoelectric components may conform to the inner surfaces of the lateral components. The force may be the force applied due to assembly, a compression force, (such as a spinal compression force) or a shear force, or a combination of such forces. The force may be about 10 N, 25 N, 50 N, 100 N, 200 N, 500 N, 700 N, 1000 N, 1500 N, 3000 N, 5000 N, or 10,000 N. The force may be variable and range between an upper and lower bound. The electrical output may include a voltage of about 0.1 mV, 0.3 mV, 0.5 mV, 1 mV, 10 mV, 25 mV, 100 mV, 200 mV, 500 mV, 700 mV, 1 V, 2 V, 3 V, 4 V, 5 V, 6 V, 7 V, 10 V, 15 V, or 20 V. The electrical voltage output may be variable and range between an upper and lower bound. The electrical output may include a current of about 50 nA, 100 nA, 500 nA, 1000 nA, 3000 nA, 5000 nA, 7000 nA, 10,000 nA, 50,000 nA, 100,000 nA, 250,000 nA, 500,000 nA, 750,000 nA, 1 mA, 5 mA, 10 mA, 15 mA, 25 mA, 35 mA, or 50 mA. The electrical current output may be variable and range between an upper and lower bound. All ranges between any of the above values are hereby disclosed.

According to some embodiments, providing the first lateral component and providing the second lateral component includes using at least one of 3D printing, machining, wire electrical discharge machining (wire EDM), laser cutting, stamping, forging, injection molding, extrusion, casting, sintering, hot isostatic pressing, or laminating or bonding of layers.

The meaning of “a,” “an,” and “the” include plural references. The meaning of “in” includes “in” and “on.”

The terms “substantially,” “close,” “approximately,” “near,” and “about,” generally refer to being within +/−10% of a target value (unless specifically specified). Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner, and are not intended to imply that the objects so described must necessarily be made of different materials or have different dimensions.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of embodiments has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The description may use perspective-based descriptions such as top/bottom, in/out, over/under, and the like. Such descriptions are merely used to facilitate the discussion and are not intended to restrict the application of embodiments described herein to any particular orientation.

As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

In embodiments, the phrase “A is located on B” means that at least a part of A is in direct physical contact or indirect physical contact (having one or more other features between A and B) with at least a part of B.

In the instant description, “A is adjacent to B” means that at least part of A is in direct physical contact with at least a part of B.

In the instant description, “B is between A and C” means that at least part of B is in or along a space separating A and C and that the at least part of B is in direct or indirect physical contact with A and C.

In the instant description, “A is attached to B” means that at least part of A is mechanically attached to at least part of B, either directly or indirectly (having one or more other features between A and B).

In the instant description, “the As are coupled to the Bs” means that at least some of the As are coupled to at least some of the Bs, and not necessarily that all As are coupled to at least one B and all Bs are coupled to at least one A.

In the instant description, “A is within B” means that at least some of A is encompassed within the physical boundaries of B.

The use of reference numerals separated by a “/”, such as “102/104” for example, is intended to refer to 102 or 104 as appropriate. Otherwise, the forward slash (“/”) as used herein means “and/or.”

When used to describe a range of dimensions, the phrase “between X and Y” represents a range that includes X and Y. Although certain elements may be referred to in the singular herein, such elements may include multiple sub-elements. For example, “an insulating material” may include one or more insulating materials. As used herein, a “conductive contact” may refer to a portion of conductive material (e.g., metal) serving as an electrical interface between different components; conductive contacts may be recessed in, flush with, or extending away from a surface of a component, and may take any suitable form (e.g., a conductive pad or socket, or portion of a conductive line or via).

In some embodiments, the techniques, processes and/or methods described herein can be detected based on the structures formed therefrom. In addition, in some embodiments, the techniques and structures described herein can be detected based on the benefits derived therefrom. Numerous configurations and variations will be apparent in light of this disclosure.

The description may use the phrases “in an embodiment,” “according to some embodiments,” “in accordance with embodiments,” or “in embodiments,” which may each refer to one or more of the same or different embodiments. Furthermore, the terms “comprising,” “including,” “having,” and the like, as used with respect to embodiments of the present disclosure, are synonymous.

The foregoing description and summary are to be understood as being in every respect illustrative and exemplary, but not restrictive, and the scope disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit.

### Examples

Illustrative examples of the technologies described throughout this disclosure are provided below. Embodiments of these technologies may include any one or more, and any combination of, the examples described below. In some embodiments, at least one of the systems or components set forth in one or more of the preceding figures may be configured as set forth in the following examples.

### EXAMPLES

Example 1 includes an orthopedic fixation assembly comprising: an elongated body comprising a first lateral component having a first inner surface, and a second lateral component having a second inner surface, the second inner surface facing and conformal with the first inner surface and defining an interface therewith, the interface defining a helicoid shape about a line extending through the elongated body; and a piezoelectric material at the interface.

Example 2 includes the subject matter of Example 1, wherein the helicoid shape defines from a fraction of a revolution to multiple revolutions about the line extending through the elongated body.

Example 3 includes the subject matter of any one of Examples 1-2, wherein the helicoid shape defines from about 0.8 revolution to about 4 revolutions about the line extending through the elongated body.

Example 4 includes the subject matter of any one of Examples 1-3, wherein the helicoid shape is continuous along an entirety of the interface.

Example 5 includes the subject matter of any one of Examples 1-3, wherein a pitch of the helicoid shape varies along a length of the line extending through the elongated body.

Example 6 includes the subject matter of Example 5, wherein the pitch is higher at one end of the line than at another end of the line.

Example 7 includes the subject matter of any one of Examples 1-3, wherein the helicoid shape includes a plurality of discontinuous helicoid shapes, individual ones of the plurality of discontinuous helicoid shapes defined about a line extending through the elongated body.

Example 8 includes the subject matter of any one of Examples 1-7, wherein the elongated body is threaded at an outer surface thereof to define an orthopedic fixation screw assembly.

Example 9 includes the subject matter of any one of Examples 1-7, wherein the elongated body is smooth at an outer surface thereof to define an orthopedic fixation rod assembly.

Example 10 includes the subject matter of any one of Examples 1-7, wherein the elongated body comprises a threaded outer surface along only a portion of its length, and a smooth outer surface along another portion of its length.

Example 11 includes the subject matter of any one of Examples 1-10, wherein the elongated body is substantially cylindrical.

Example 12 includes the subject matter of any one of Examples 1-11, wherein the elongated body comprises a cross-section selected from the group consisting of oval, polygonal, rectangular, square, and star-shaped.

Example 13 includes the subject matter of any one of Examples 1-12, wherein the elongated body further defines a lumen extending therethrough, the lumen configured to receive a guidewire to guide the orthopedic fixation assembly during implantation.

Example 14 includes the subject matter of Example 13, wherein the piezoelectric material is configured to allow the lumen to remain unobstructed for passage of the guidewire.

Example 15 includes the subject matter of any one of Examples 1-12, wherein the interface defines a non-planar profile to distribute at least one of compressive forces or shear forces unevenly to the piezoelectric material.

Example 16 includes the subject matter of Example 15, wherein the non-planar profile includes a wavelike configuration.

Example 17 includes the subject matter of any one of Examples 15-16, wherein the non-planar profile at least one of is in a plane transverse to the line extending through the elongated body or is defined in a direction of the line extending through the elongated body.

Example 18 includes the subject matter of any one of Examples 1-17, wherein the line corresponds to a straight longitudinal line extending through the elongated body.

Example 19 includes the subject matter of any one of Examples 1-18, wherein the first lateral component and the second lateral component each comprise an electrically conductive material.

Example 20 includes the subject matter of any one of Examples 1-19, wherein the first lateral component and the second lateral component are configured to interlock with one another.

Example 21 includes the subject matter of any one of Examples 1-20, wherein the first lateral component and the second lateral component include titanium.

Example 22 includes the subject matter of any one of Examples 1-21, wherein outer surfaces of the first lateral component and of the second lateral component are porous to promote bone growth.

Example 23 includes the subject matter of any one of Examples 1-22, further comprising a fastener configured to fasten the first lateral component and the second lateral component to one another.

Example 24 includes the subject matter of Example 23, wherein the fastener comprises an electrically insulating component.

Example 25 includes the subject matter of Example 24, wherein the electrically insulating component comprises PEEK.

Example 26 includes the subject matter of Example 25, wherein the fastener further comprises an electrically conductive component.

Example 27 includes the subject matter of any one of Examples 23-26, wherein the fastener is threaded or configured for a press fit fastening of the first lateral component and the second lateral component together.

Example 28 includes the subject matter of any one of Examples 23-27, wherein the fastener comprises a screw.

Example 29 includes the subject matter of any one of Examples 23-28, wherein the fastener comprises an electrically conductive threaded component and an electrically isolating washer, the washer electrically insulating the threaded component from the first lateral component and from the second lateral component.

Example 30 includes the subject matter of any one of Examples 23-29, wherein the fastener is a fastener cap provided at one of a proximal end or a distal end of the elongated body.

Example 31 includes the subject matter of Example 30, wherein the fastener cap is a first fastener cap, the orthopedic fixation assembly further including a second fastener cap provided at another one of the proximal end or the distal end of the elongated body.

Example 32 includes the subject matter of Example 30, wherein the fastener cap comprises an outer surface that is recessed with respect to, or protrudes outward from, an outermost surface of the elongated body.

Example 33 includes the subject matter of Example 30, wherein the fastener cap comprises longitudinal splines and grooves configured for a press fit with a splined head of the elongated body.

Example 34 includes the subject matter of any one of Examples 23-30, wherein the fastener comprises a washer portion having protrusions configured to engage bone material.

Example 35 includes the subject matter of any one of Examples 1-34, wherein the piezoelectric material comprises one or more piezoelectric elements.

Example 36 includes the subject matter of any one of Examples 1-35, wherein the piezoelectric material comprises a piezoelectric sheet.

Example 37 includes the subject matter of Example 36, wherein the piezoelectric sheet extends across an entirety of an interface defined between the first inner surface and the second inner surface.

Example 38 includes the subject matter of any one of Examples 1-37, wherein the piezoelectric material comprises a polymer selected from the group consisting of polyvinylidene fluoride (PVDF), polyvinylidene fluoride-trifluoroethylene (PVDF-TrFE), or combinations thereof.

Example 39 includes the subject matter of any one of Examples 1-38, wherein the piezoelectric material comprises a matrix including piezoelectric fillers selected from the group consisting of piezoelectric ceramic fibers, rods, particles, or single-crystal whiskers.

Example 40 includes a kit to form an orthopedic fixation assembly, the kit comprising: a first lateral component having a first inner surface defining a first helicoid shape; a second lateral component having a second inner surface defining a second helicoid shape, wherein the first lateral component and the second lateral component are configured to be assembled together to form an elongated body, wherein, in the elongated body, the second inner surface faces the first inner surface and the first helicoid shape and the second helicoid shape define an interface therebetween, the interface defining an interfacial helicoid shape about a line extending through the elongated body; and a piezoelectric material configured to be placed in the elongated body at the interface.

Example 41 includes the subject matter of Example 40, wherein the first inner surface and the second inner surface are configured such that the interfacial helicoid shape defines from a fraction of a revolution to multiple revolutions about the line extending through the elongated body.

Example 42 includes the subject matter of any one of Examples 40-41, wherein the first inner surface and the second inner surface are configured such that the interfacial helicoid shape defines from about 0.8 revolution to about 4 revolutions about the line extending through the elongated body.

Example 43 includes the subject matter of any one of Examples 40-42, wherein the first inner surface and the second inner surface are configured such that the interfacial helicoid shape is continuous along an entirety of the interface.

Example 44 includes the subject matter of any one of Examples 40-42, wherein a pitch of the helicoid shape varies along a length of the line extending through the elongated body.

Example 45 includes the subject matter of Example 44, wherein the pitch is higher at one end of the line than at another end of the line.

Example 46 includes the subject matter of any one of Examples 40-42, wherein the first inner surface and the second inner surface are configured such that the interfacial helicoid shape includes a plurality of discontinuous helicoid shapes, individual ones of the plurality of discontinuous helicoid shapes defined about a line extending through the elongated body.

Example 47 includes the subject matter of any one of Examples 40-46, wherein the first lateral component and the second lateral component include thread configurations at respective outer surfaces thereof such that the elongated body is threaded at an outer surface thereof to define an orthopedic fixation screw assembly.

Example 48 includes the subject matter of any one of Examples 40-46, wherein the first lateral component and the second lateral component have smooth respective outer surfaces such that the elongated body is smooth at an outer surface thereof to define an orthopedic fixation rod assembly.

Example 49 includes the subject matter of any one of Examples 40-46, wherein the elongated body comprises a threaded outer surface along only a portion of its length, and a smooth outer surface along another portion of its length.

Example 50 includes the subject matter of any one of Examples 40-49, wherein the first lateral component and the second lateral component are configured such that the elongated body is substantially cylindrical.

Example 51 includes the subject matter of any one of Examples 40-50, wherein the first lateral component and the second lateral component are configured such that the elongated body comprises a cross-section selected from the group consisting of oval, polygonal, rectangular, square, and star-shaped.

Example 52 includes the subject matter of any one of Examples 40-51, wherein the first lateral component and the second lateral component define respective longitudinal grooves at corresponding ones of the first inner surface and the second inner surface, the grooves configured such that the elongated body further defines a longitudinal lumen formed by a registration of respective ones of the grooves the lumen configured to receive a guidewire to guide the orthopedic fixation assembly during implantation.

Example 53 includes the subject matter of Example 52, wherein the piezoelectric material is configured to allow the lumen to remain unobstructed for passage of the guidewire.

Example 54 includes the subject matter of any one of Examples 40-51, wherein the first inner surface and the second inner surface define respective non-planar profiles such that the interface defines an interfacial non-planar profile to distribute at least one of compressive forces or shear forces unevenly to the piezoelectric material.

Example 55 includes the subject matter of Example 54, wherein the interfacial non-planar profile includes a wavelike configuration.

Example 56 includes the subject matter of any one of Examples 54-55, wherein the first inner surface and the second inner surface are configured such that the interfacial non-planar profile at least one of is in a plane transverse to the line extending through the elongated body or is defined in a direction of the line extending through the elongated body.

Example 57 includes the subject matter of any one of Examples 40-56, wherein the line corresponds to a straight longitudinal line extending through the elongated body.

Example 58 includes the subject matter of any one of Examples 40-57, wherein the first lateral component and the second lateral component each comprise an electrically conductive material.

Example 59 includes the subject matter of any one of Examples 40-58, wherein the first lateral component and the second lateral component are configured to interlock with one another.

Example 60 includes the subject matter of any one of Examples 40-59, wherein the first lateral component and the second lateral component include titanium.

Example 61 includes the subject matter of any one of Examples 40-60, wherein outer surfaces of the first lateral component and of the second lateral component are porous to promote bone growth.

Example 62 includes the subject matter of any one of Examples 40-61, further comprising a fastener configured to fasten the first lateral component and the second lateral component to one another.

Example 63 includes the subject matter of Example 62, wherein the fastener comprises an electrically insulating component.

Example 64 includes the subject matter of Example 63, wherein the electrically insulating component comprises PEEK.

Example 65 includes the subject matter of Example 64, wherein the fastener further comprises an electrically conductive component.

Example 66 includes the subject matter of any one of Examples 62-65, wherein the fastener is threaded or configured for a press fit fastening of the first lateral component and the second lateral component together.

Example 67 includes the subject matter of any one of Examples 62-66, wherein the fastener comprises a screw.

Example 68 includes the subject matter of any one of Examples 62-67, wherein the fastener comprises an electrically conductive threaded component and an electrically isolating washer, the washer electrically insulating the threaded component from the first lateral component and from the second lateral component.

Example 69 includes the subject matter of any one of Examples 62-68, wherein the fastener is a fastener cap provided at one of a proximal end or a distal end of the elongated body.

Example 70 includes the subject matter of Example 69, wherein the fastener cap is a first fastener cap, the kit further including a second fastener cap provided at another one of the proximal end or the distal end of the elongated body.

Example 71 includes the subject matter of Example 69, wherein the fastener cap comprises an outer surface that, in the orthopedic fixation assembly, is recessed with respect to, or protrudes outward from, an outermost surface of the elongated body.

Example 72 includes the subject matter of Example 69, wherein the fastener cap comprises longitudinal splines and grooves configured for a press fit with a splined head of the elongated body.

Example 73 includes the subject matter of any one of Examples 62-69, wherein the fastener comprises a washer portion having protrusions configured to engage bone material.

Example 74 includes the subject matter of any one of Examples 40-73, wherein the piezoelectric material comprises one or more piezoelectric elements.

Example 75 includes the subject matter of any one of Examples 40-74, wherein the piezoelectric material comprises a piezoelectric sheet.

Example 76 includes the subject matter of Example 75, wherein the piezoelectric sheet extends across an entirety of an interface defined between the first inner surface and the second inner surface.

Example 77 includes the subject matter of any one of Examples 40-76, wherein the piezoelectric material comprises a polymer selected from the group consisting of polyvinylidene fluoride (PVDF), polyvinylidene fluoride-trifluoroethylene (PVDF-TrFE), or combinations thereof.

Example 78 includes the subject matter of any one of Examples 40-77, wherein the piezoelectric material comprises a matrix including piezoelectric fillers selected from the group consisting of piezoelectric ceramic fibers, rods, particles, or single-crystal whiskers.

Example 79 includes the subject matter of any one of Examples 1-78, wherein the orthopedic fixation assembly is configured such that, during cyclic loading and unloading, the piezoelectric material generates an alternating current, with the polarity of the generated charge reversing as the load is applied and released.

Example 80 includes the subject matter of any one of Examples 1-79, wherein the piezoelectric material is configured and positioned to biomimetically replicate the natural piezoelectric signaling of bone, thereby promoting bone growth and remodeling in response to physiological mechanical stress.

Example 81 includes the subject matter of any one of Examples 1-80, wherein at least one of the first lateral component or the second lateral component comprises a surface texture or roughness that is characteristic of a specific manufacturing method, such as additive manufacturing or wire electrical discharge machining.

Example 82 includes the subject matter of any one of Examples 1-81, wherein the piezoelectric material is provided in a discontinuous or segmented form, such as a plurality of strips, segments, or discrete elements, to facilitate assembly or to accommodate a central lumen.

Example 83 includes the subject matter of any one of Examples 1-82, wherein the number of helicoid revolutions of the interface is selected based on the intended clinical application, such that a greater number of revolutions is provided for implants subject to bending loads and a lesser number for implants subject to compressive loads.

Example 84 includes the subject matter of any one of Examples 1-83, wherein the self-interlocking property of the assembly is enhanced by increasing the number of helicoid revolutions, such that the lateral components are increasingly resistant to separation as the number of revolutions increases.

Example 85 includes the subject matter of any one of Examples 1-84, wherein the orthopedic fixation assembly is provided as a modular kit or as a blank helical construct that can be further machined or finished for specific applications, including the addition of threads, surface features, or attachment points.

Example 86 includes the subject matter of any one of Examples 1-85, wherein the orthopedic fixation assembly is dimensioned and configured to be compatible with existing orthopedic systems, such as pedicle screw tulips, trauma plates, or other standard fixation hardware.

Example 87 includes the subject matter of any one of Examples 1-86, wherein the outer surfaces of the first lateral component and/or the second lateral component are intentionally roughened, textured, or made porous to promote bone ongrowth and osseointegration, and wherein the specific surface characteristics are selected based on the intended application or manufacturing method.

Example 88 includes the subject matter of any one of Examples 1-87, wherein the fastener cap is selected from a press fit cap or a threaded cap, and wherein the type of cap is chosen based on the desired balance between ease of assembly and fixation strength.

Example 89 includes the subject matter of any one of Examples 1-88, wherein the assembly further comprises a washer portion with protrusions, such as teeth or spikes, configured to engage the surrounding bone and distribute load over a larger surface area.

Example 90 includes the subject matter of any one of Examples 1-89, wherein the piezoelectric material is provided in a split or bifurcated configuration, with piezoelectric elements positioned on either side of a central lumen to maintain electrical functionality while preserving an unobstructed passage for a guidewire.

Example 91 includes the subject matter of any one of Examples 1-90, wherein the pitch of the helicoid interface varies along the length of the implant, such that a higher pitch is provided at one end to facilitate assembly or insertion, and a lower pitch is provided at the other end to enhance mechanical interlocking and load transfer.

Example 92 includes the subject matter of any one of Examples 1-91, wherein the orthopedic fixation assembly is configured such that the alternating current generated by the piezoelectric material under cyclic loading is delivered to the surrounding bone tissue to enhance osteogenic signaling.

Example 93 includes the subject matter of any one of Examples 1-92, wherein the modular kit includes interchangeable fastener caps, washers, or other components, allowing the assembly to be adapted intraoperatively to the patient's anatomy or the clinical scenario.

Example 94 includes the subject matter of any one of Examples 1-93, wherein the surface texture or roughness of the lateral components is quantified by a surface roughness parameter, and wherein the surface roughness is selected to optimize bone ongrowth or to provide a distinguishing structural feature indicative of the manufacturing method.

Example 95 includes the subject matter of any one of Examples 1-94, wherein the orthopedic fixation assembly is provided as a blank that can be machined to include threads, splines, or other features compatible with a range of orthopedic fixation systems.

Example 96 includes the subject matter of any one of Examples 1-95, wherein the piezoelectric material is arranged in a pattern or configuration that maximizes electrical output in regions of highest mechanical stress, such as at the interface between the lateral components in areas subject to bending or torsion.

Example 97 includes the subject matter of any one of Examples 1-96, wherein the orthopedic fixation assembly is configured such that the self-interlocking property is sufficient to maintain assembly integrity under physiological loads without the need for additional fasteners in certain clinical applications.

Example 98 includes the subject matter of any one of Examples 1-97, wherein the orthopedic fixation assembly is provided with a surface finish or coating that is selected to enhance biocompatibility, reduce friction during insertion, or provide antimicrobial properties.

Example 99 includes the subject matter of any one of Examples 1-98, wherein the modular kit includes a set of piezoelectric elements of varying thicknesses, lengths, or material compositions, allowing the surgeon to select the optimal element for the specific clinical application.

Example 100 includes the subject matter of any one of Examples 1-99, wherein the orthopedic fixation assembly is configured such that the alternating current generated by the piezoelectric material is monitored or measured intraoperatively or postoperatively to assess implant function or bone healing progress.

Example 101 includes a method of using an orthopedic fixation assembly, the method comprising: providing an orthopedic fixation assembly comprising an elongated body having a first lateral component with a first inner surface, a second lateral component with a second inner surface facing and conformal with the first inner surface and defining an interface therewith, the interface defining a helicoid shape about a line extending through the elongated body, and a piezoelectric material at the interface; positioning the orthopedic fixation assembly at a target bone site; and implanting the orthopedic fixation assembly into bone such that the elongated body traverses or anchors to the bone.

Example 102 includes the subject matter of Example 101, further comprising guiding the orthopedic fixation assembly to the target bone site using a guidewire received within a lumen defined by the elongated body.

Example 103 includes the subject matter of any one of Examples 101-102, wherein the orthopedic fixation assembly is implanted as a screw by rotating the assembly into bone using threads on an outer surface of the elongated body.

Example 104 includes the subject matter of any one of Examples 101-103, wherein the orthopedic fixation assembly is implanted as a rod by press fitting or sliding the assembly into a medullary space.

Example 105 includes the subject matter of any one of Examples 101-104, further comprising securing the first lateral component and the second lateral component together with a fastener selected from the group consisting of a screw, a threaded cap, a press fit cap, or a washer.

Example 106 includes a method of making an orthopedic fixation assembly, the method comprising: providing a first lateral component having a first inner surface; providing a second lateral component having a second inner surface, the second inner surface facing and conformal with the first inner surface; providing a piezoelectric material; assembling the first lateral component and the second lateral component together such that the first inner surface and the second inner surface define a helicoid interface about a line extending through the elongated body, with the piezoelectric material positioned at the interface; and securing the first lateral component and the second lateral component together to form an elongated body.

Example 107 includes the subject matter of Example 106, wherein securing comprises fastening the first lateral component and the second lateral component together with a fastener selected from the group consisting of a screw, a threaded cap, or a press fit cap.

Example 108 includes the subject matter of any one of Examples 106-107, further comprising forming a lumen extending through the elongated body by aligning grooves in the first inner surface and the second inner surface.

Example 109 includes the subject matter of any one of Examples 106-108, further comprising forming threads on an outer surface of the elongated body to define an orthopedic fixation screw assembly.

Example 110 includes the subject matter of any one of Examples 106-109, further comprising forming a non-planar profile at the interface to distribute compressive or shear forces unevenly to the piezoelectric material.

Example 111 includes the subject matter of any one of Examples 101-110, wherein providing the first lateral component and providing the second lateral component includes using at least one of 3D printing, machining, wire electrical discharge machining (wire EDM), laser cutting, stamping, forging, injection molding, extrusion, casting, sintering, hot isostatic pressing, or laminating or bonding of layers.

Example 112 includes a method of making a kit for forming an orthopedic fixation assembly, the method comprising: providing a first lateral component having a first inner surface defining a first helicoid shape; providing a second lateral component having a second inner surface defining a second helicoid shape, the second inner surface configured to face and conform with the first inner surface when assembled; providing a piezoelectric material configured to be placed at an interface defined between the first inner surface and the second inner surface; and packaging the first lateral component, the second lateral component, and the piezoelectric material together as a kit.

Example 113 includes the subject matter of Example 112, further comprising including in the kit a fastener configured to fasten the first lateral component and the second lateral component together.

Example 114 includes the subject matter of any one of Examples 112-113, further comprising forming grooves in the first inner surface and the second inner surface, the grooves configured to define a lumen for a guidewire when the components are assembled.

Example 115 includes the subject matter of any one of Examples 112-114, further comprising selecting the piezoelectric material from the group consisting of a piezoelectric sheet, a plurality of piezoelectric elements, or a matrix including piezoelectric fillers.

Example 116 includes the subject matter of any one of Examples 112-115, further comprising including in the kit at least one fastener cap, washer, or additional modular component for assembly customization.

What is claimed is:

1. An orthopedic fixation assembly comprising:
an elongated body comprising a first lateral component having a first inner surface, and a second lateral component having a second inner surface, the second inner surface facing and conformal with the first inner surface and defining an interface therewith, the interface defining a helicoid shape about a line extending through the elongated body; and
a piezoelectric material at the interface.

2. The orthopedic fixation assembly of claim 1, wherein the helicoid shape defines in total from a fraction of a revolution to multiple revolutions about the line extending through the elongated body.

3. The orthopedic fixation assembly of claim 2, wherein the helicoid shape defines in total from about 0.8 revolution to about 4 revolutions about the line extending through the elongated body.

4. The orthopedic fixation assembly of claim 1, wherein the helicoid shape is continuous along an entirety of the interface.

5. The orthopedic fixation assembly of claim 1, wherein a pitch of the helicoid shape varies along a length of the line extending through the elongated body.

6. The orthopedic fixation assembly of claim 5, wherein the pitch is higher at one end of the line than at another end of the line.

7. The orthopedic fixation assembly of claim 1, wherein the helicoid shape includes a plurality of discontinuous helicoid shapes, individual ones of the plurality of discontinuous helicoid shapes defined about a line extending through the elongated body.

8. The orthopedic fixation assembly of claim 1, wherein the elongated body is threaded at an outer surface thereof to define an orthopedic fixation screw assembly.

9. The orthopedic fixation assembly of claim 1, wherein the elongated body is smooth at an outer surface thereof to define an orthopedic fixation rod assembly.

10. The orthopedic fixation assembly of claim 1, wherein the elongated body comprises a threaded outer surface along only a portion of its length, and a smooth outer surface along another portion of its length.

11. The orthopedic fixation assembly of claim 1, wherein the elongated body further defines a lumen extending therethrough, the lumen configured to receive a guidewire to guide the orthopedic fixation assembly during implantation.

12. The orthopedic fixation assembly of claim 1, wherein the interface defines a non-planar profile to distribute at least one of compressive forces or shear forces unevenly to the piezoelectric material.

13. The orthopedic fixation assembly of claim 1, wherein the line corresponds to a straight longitudinal line extending through the elongated body.

14. The orthopedic fixation assembly of claim 1, wherein the first lateral component and the second lateral component each comprise an electrically conductive material.

15. The orthopedic fixation assembly of claim 1, wherein the first lateral component and the second lateral component are configured to interlock with one another.

16. The orthopedic fixation assembly of claim 1, wherein outer surfaces of the first lateral component and of the second lateral component are porous to promote bone growth.

17. The orthopedic fixation assembly of claim 1, further comprising a fastener configured to fasten the first lateral component and the second lateral component to one another.

18. The orthopedic fixation assembly of claim 17, wherein the fastener comprises an electrically insulating component.

19. The orthopedic fixation assembly of claim 18, wherein the electrically insulating component comprises PEEK.

20. The orthopedic fixation assembly of claim 19, wherein the fastener further comprises an electrically conductive component.

21. The orthopedic fixation assembly of claim 17, wherein the fastener is threaded or configured for a press fit fastening of the first lateral component and the second lateral component together.

22. The orthopedic fixation assembly of claim 17, wherein the fastener comprises a screw.

23. The orthopedic fixation assembly of claim 17, wherein the fastener comprises an electrically conductive threaded component and an electrically isolating washer, the washer electrically insulating the threaded component from the first lateral component and from the second lateral component.

24. The orthopedic fixation assembly of claim 17, wherein the fastener is a fastener cap provided at one of a proximal end or a distal end of the elongated body.

25. The orthopedic fixation assembly of claim 24, wherein the fastener cap is a first fastener cap, the orthopedic fixation assembly further including a second fastener cap provided at another one of the proximal end or the distal end of the elongated body.

26. The orthopedic fixation assembly of claim 24, wherein the fastener cap comprises longitudinal splines and grooves configured for a press fit with a splined head of the elongated body.

27. The orthopedic fixation assembly of claim 17, wherein the fastener comprises a washer portion having protrusions configured to engage bone material.

28. The orthopedic fixation assembly of claim 1, wherein the piezoelectric material comprises one or more piezoelectric elements.

29. The orthopedic fixation assembly of claim 1, wherein the piezoelectric material comprises a piezoelectric sheet.

30. The orthopedic fixation assembly of claim 29, wherein the piezoelectric sheet extends across an entirety of an interface defined between the first inner surface and the second inner surface.

31. The orthopedic fixation assembly of claim 1, wherein the piezoelectric material comprises a polymer selected from the group consisting of polyvinylidene fluoride (PVDF), polyvinylidene fluoride-trifluoroethylene (PVDF-TrFE), or combinations thereof.

32. A kit to form an orthopedic fixation assembly, the kit comprising:
a first lateral component having a first inner surface defining a first helicoid shape;
a second lateral component having a second inner surface defining a second helicoid shape, wherein the first lateral component and the second lateral component are configured to be assembled together to form an elongated body, wherein, in the elongated body, the second inner surface faces the first inner surface and the first helicoid shape and the second helicoid shape define an interface therebetween, the interface defining an interfacial helicoid shape about a line extending through the elongated body; and
a piezoelectric material configured to be placed in the elongated body at the interface.

33. The kit of claim 32, wherein the first inner surface and the second inner surface are configured such that the interfacial helicoid shape defines in total from a fraction of a revolution to multiple revolutions about the line extending through the elongated body.

34. The kit of claim 32, wherein the first inner surface and the second inner surface are configured such that the interfacial helicoid shape is continuous along an entirety of the interface.

35. The kit of claim 32, wherein a pitch of the helicoid shape varies along a length of the line extending through the elongated body.

36. The kit of claim 35, wherein the pitch is higher at one end of the line than at another end of the line.

37. The kit of claim 32, wherein the first inner surface and the second inner surface are configured such that the interfacial helicoid shape includes a plurality of discontinuous helicoid shapes, individual ones of the plurality of discontinuous helicoid shapes defined about a line extending through the elongated body.

38. The kit of claim 32, wherein the first lateral component and the second lateral component include thread configurations at respective outer surfaces thereof such that the elongated body is threaded at an outer surface thereof to define an orthopedic fixation screw assembly.

39. The kit of claim 32, wherein the first lateral component and the second lateral component have smooth respective outer surfaces such that the elongated body is smooth at an outer surface thereof to define an orthopedic fixation rod assembly.

40. The kit of claim 32, wherein the elongated body comprises a threaded outer surface along only a portion of its length, and a smooth outer surface along another portion of its length.

41. The kit of claim 32, wherein the first lateral component and the second lateral component define respective longitudinal grooves at corresponding ones of the first inner surface and the second inner surface, the grooves configured such that the elongated body further defines a longitudinal lumen formed by a registration of respective ones of the grooves, the lumen configured to receive a guidewire to guide the orthopedic fixation assembly during implantation.

42. The kit of claim 32, wherein the line corresponds to a straight longitudinal line extending through the elongated body.

43. The kit of claim 32, wherein the first lateral component and the second lateral component each comprise an electrically conductive material.

44. The kit of claim 32, wherein outer surfaces of the first lateral component and of the second lateral component are porous to promote bone growth.

45. The kit of claim 32, further comprising a fastener configured to fasten the first lateral component and the second lateral component to one another.

46. The kit of claim 45, wherein the fastener is threaded or configured for a press fit fastening of the first lateral component and the second lateral component together.

47. The kit of claim 45, wherein the fastener comprises an electrically conductive threaded component and an electrically isolating washer, the washer electrically insulating the threaded component from the first lateral component and from the second lateral component.

48. The kit of claim 32, wherein the piezoelectric material comprises one or more piezoelectric elements.

49. A method of making an orthopedic fixation assembly, the method comprising: providing a first lateral component having a first inner surface; providing a second lateral component having a second inner surface, the second inner surface facing and conformal with the first inner surface; providing a piezoelectric material; assembling the first lateral component and the second lateral component together to form an elongated body such that the first inner surface and the second inner surface define a helicoid interface about a line extending through the elongated body, with the piezoelectric material positioned at the interface; and securing the first lateral component and the second lateral component together to form an elongated body.

50. The method of claim 49, wherein securing comprises fastening the first lateral component and the second lateral component together with a fastener selected from the group consisting of a screw, a threaded cap, or a press fit cap.

51. The method of claim 49, further comprising forming a lumen extending through the elongated body by aligning grooves in the first inner surface and the second inner surface.

52. The method of claim 49, further comprising forming threads on an outer surface of the elongated body to define an orthopedic fixation screw assembly.

53. The method of claim 49, wherein providing the first lateral component and providing the second lateral component includes using at least one of 3D printing, machining, wire electrical discharge machining (wire EDM), laser cutting, stamping, forging, injection molding, extrusion, casting, sintering, hot isostatic pressing, or laminating or bonding of layers.
